SEQUENCE LISTING (42 sequences)

SEQ ID NO: 1 - DNA, 6389 bp, Adenovirus 5
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagcaccatgcatcatcaccatcaccatatgggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagggatccatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacgcaggaaacaggagacacaactccaagtgcatactctatgtcattttcatgggactggtctggccacaactacattaatgaaatatttgccacatcctcttacactttttcatacattgcccaagaataaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 2 - DNA, 6448 bp, Adenovirus 5
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagcaccatgcaccaccaccatcaccacggccctaaaaagaagcgtaaagtcggcggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagggcgtcgtccgtcgacgtggatcaggatcaggatccggttccggttccatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcggccgctctagaccaggccctggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 3 - DNA, 6181 bp, Adenovirus 5
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagcaccatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacgcaggaaacaggagacacaactccaagtgcatactctatgtcattttcatgggactggtctggccacaactacattaatgaaatatttgccacatcctcttacactttttcatacattgcccaagaaggatcaggatcaggatcaggatcaggatcacatcatcaccatcaccattaagcggccgctctagaccaggccctggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - DNA, 5861 bp, Adenovirus 5
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacacgtgtgatcagatatcgccaccatgcaccaccaccatcaccacggccctaaaaagaagcgtaaagtcggcggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggagggcgtcgtccgtcgacgtggatcaggatcaggatccggttccggttcctccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaatctagaccaggccctggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 5 - protein, 368 aa, Adenovirus
MSKRKIKEEMLQVIAPEIYGPPKKEEQDYKPRKLKRVKKKKKDDDDELDDEVELLHATAPRRRVQWKGRRVKRVLRPGTTVVFTPGERSTRTYKRVYDEVYGDEDLLEQANERLGEFAYGKRHKDMLALPLDEGNPTPSLKPVTLQQVLPALAPSEEKRGLKRESGDLAPTVQLMVPKRQRLEDVLEKMTVEPGLEPEVRVRPIKQVAPGLGVQTVDVQIPTTSSTSIATATEGMETQTSPVASAVADAAVQAVAAAASKTSTEVQTDPWMFRVSAPRRPRGSRKYGAASALLPEYALHPSIAPTPGYRGYTYRPRRRATTRRRTTTGTRRRRRRRQPVLAPISVRRVAREGGRTLVLPTARYHPSIV

SEQ ID NO: 6 - DNA, 1107 bp, Adenovirus 5
atgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaa

SEQ ID NO: 7 - protein, 83 aa, Artificial Sequence
  HMG box A
GKGDPKKPRGKMSSYAFFVQTCREEHKKKHPDASVNFSEFSKKCSERWKTMSAKEKGKFEDMAKADKARYEREMKTYIPPKGE

SEQ ID NO: 8 - DNA, 249 bp, Artificial Sequence
  HMG box A
ggcaaaggagatcctaagaagccgagaggcaaaatgtcatcatatgcattttttgtgcaaacttgtcgggaggagcataagaagaagcacccagatgcttcagtcaacttctcagagttttctaagaagtgctcagagaggtggaagaccatgtctgctaaagagaaaggaaaatttgaagatatggcaaaagcggacaaggcccgttatgaaagagaaatgaaaacctatatccctcccaaaggggag

SEQ ID NO: 9 - protein, 473 aa, Artificial Sequence
  HMG-V
MHHHHHHGPKKKRKVGGKGDPKKPRGKMSSYAFFVQTCREEHKKKHPDASVNFSEFSKKCSERWKTMSAKEKGKFEDMAKADKARYEREMKTYIPPKGEGGSGSGSSKRKIKEEMLQVIAPEIYGPPKKEEQDYKPRKLKRVKKKKKDDDDELDDEVELLHATAPRRRVQWKGRRVKRVLRPGTTVVFTPGERSTRTYKRVYDEVYGDEDLLEQANERLGEFAYGKRHKDMLALPLDEGNPTPSLKPVTLQQVLPALAPSEEKRGLKRESGDLAPTVQLMVPKRQRLEDVLEKMTVEPGLEPEVRVRPIKQVAPGLGVQTVDVQIPTTSSTSIATATEGMETQTSPVASAVADAAVQAVAAAASKTSTEVQTDPWMFRVSAPRRPRGSRKYGAASALLPEYALHPSIAPTPGYRGYTYRPRRRATTRRRTTTGTRRRRRRRQPVLAPISVRRVAREGGRTLVLPTARYHPSIV

SEQ ID NO: 10 - DNA, 50 bp, Artificial Sequence
  oligonucleotide
ctgcagcaccatgcatcatcaccatcaccatatgggcaaaggagatccta

SEQ ID NO: 11 - DNA, 50 bp, Artificial Sequence
  oligonucleotide
catatgatgacattttgcctctcggcttcttaggatctcctttgcccata

SEQ ID NO: 12 - DNA, 50 bp, Artificial Sequence
  oligonucleotide
aggcaaaatgtcatcatatgcattttttgtgcaaacttgtcgggaggagc

SEQ ID NO: 13 - DNA, 50 bp, Artificial Sequence
  oligonucleotide
tgactgaagcatctgggtgcttcttcttatgctcctcccgacaagtttgc

SEQ ID NO: 14 - DNA, 50 bp, Artificial Sequence
  oligonucleotide
gcacccagatgcttcagtcaacttctcagagttttctaagaagtgctcag

SEQ ID NO: 15 - DNA, 50 bp, Artificial Sequence
  oligonucleotide
tctctttagcagacatggtcttccacctctctgagcacttcttagaaaac

SEQ ID NO: 16 - DNA, 50 bp, Artificial Sequence
  oligonucleotide
gaccatgtctgctaaagagaaaggaaaatttgaagatatggcaaaagcgg

SEQ ID NO: 17 - DNA, 50 bp, Artificial Sequence
  oligonucleotide
ttttcatttctctttcataacgggccttgtccgcttttgccatatcttca

SEQ ID NO: 18 - DNA, 50 bp, Artificial Sequence
  oligonucleotide
ttatgaaagagaaatgaaaacctatatccctcccaaaggggagggatcca

SEQ ID NO: 19 - DNA, 50 bp, Artificial Sequence
  oligonucleotide
aggtatcttcagacggtcttgcgcgcttcatggatccctcccctttggga

SEQ ID NO: 20 - DNA, 50 bp, Artificial Sequence
  oligonucleotide
aagaccgtctgaagataccttcaaccccgtgtatccatatgacacggaaa

SEQ ID NO: 21 - DNA, 50 bp, Artificial Sequence
  oligonucleotide
gagtaagaaaaggcacagttggaggaccggtttccgtgtcatatggatac

SEQ ID NO: 22 - DNA, 75 bp, Artificial Sequence
  oligonucleotide
aaaagtcgaccactaaacggtacacaggaaacagggtctagaggatttaaatctggatcctacccctacgacgtg

SEQ ID NO: 23 - DNA, 75 bp, Artificial Sequence
  oligonucleotide
gaaaatgacatagagtatgcacttggagttgtgtcgccggcgtagtcgggcacgtcgtaggggtaggatccagat

SEQ ID NO: 24 - DNA, 75 bp, Artificial Sequence
  oligonucleotide
caagtgcatactctatgtcattttcatgggactggtctggccacaactacattaatgaaatatttgccacatcct

SEQ ID NO: 25 - DNA, 75 bp, Artificial Sequence
  oligonucleotide
gagcagagctttcttactgctttcttgggcaatgtatgaaaaagtgtaagaggatgtggcaaatatttcattaat

SEQ ID NO: 26 - DNA, 75 bp, Artificial Sequence
  oligonucleotide
agaaagcagtaagaaagctctgctcgccctggctttgcaccatcttgctcatctcgccttgcatcttgctcttgc

SEQ ID NO: 27 - DNA, 75 bp, Artificial Sequence
  oligonucleotide
ttgcggccgctcaatggtgatggtgatgatgactaccagccttcttcagtgcaagagcaagatgcaaggcgagat

SEQ ID NO: 28 - DNA, 85 bp, Artificial Sequence
  oligonucleotide
ctgcagcaccatgcatcatcaccatcaccatatggccaggtacagatgctgtcgcagccagagccggagcagatattaccgccag

SEQ ID NO: 29 - DNA, 75 bp, Artificial Sequence
  oligonucleotide
ctcctccgtgtctggcagctccgcctccttcgtctgcgacttctttgtctctggcggtaatatctgctccggctc

SEQ ID NO: 30 - DNA, 75 bp, Artificial Sequence
  oligonucleotide
ggcggagctgccagacacggaggagagccatgaggtgctgccgccccaggtacagaccgagatgtagaagacacg

SEQ ID NO: 31 - DNA, 97 bp, Artificial Sequence
  oligonucleotide
gaccggtttccgtgtcatatggatacacggggttgaaggtatcttcagacggtcttgcgcgcttcatggatccgtgtcttctacatctcggtctgta

SEQ ID NO: 32 - DNA, 43 bp, Artificial Sequence
  oligonucleotide
aaaggatccggttccggttccatgcggcgcgcggcgatgtatg

SEQ ID NO: 33 - DNA, 39 bp, Artificial Sequence
  oligonucleotide
taaatctagattaaaaagtgcggctcgataggacgcgcg

SEQ ID NO: 34 - DNA, 38 bp, Artificial Sequence
  oligonucleotide
ggatccggttccggttcctccaagcgcaaaatcaaaga

SEQ ID NO: 35 - DNA, 35 bp, Artificial Sequence
  oligonucleotide
tttctagattaaacgatgctggggtggtagcgcgc

SEQ ID NO: 36 - DNA, 30 bp, Artificial Sequence
  oligonucleotide
gggctccggagtgaccattctggtgagccg

SEQ ID NO: 37 - DNA, 30 bp, Artificial Sequence
  oligonucleotide
ccaggccggtctgggtgctgcggctcacca

SEQ ID NO: 38 - DNA, 30 bp, Artificial Sequence
  oligonucleotide
accggcctgggccattttacccgcagcacc

SEQ ID NO: 39 - DNA, 30 bp, Artificial Sequence
  oligonucleotide
atatcgttctggctggtctgggtgctgcgg

SEQ ID NO: 40 - DNA, 30 bp, Artificial Sequence
  oligonucleotide
agaacgatatttttgtcgtccgtcgacgtg

SEQ ID NO: 41 - DNA, 30 bp, Artificial Sequence
  oligonucleotide
accggatcctgatcctgatccacgtcgacg

SEQ ID NO: 42 - protein, 10 aa, Artificial Sequence
  V3 peptide
RGPGRAFVTI

(19) United States
(12) Patent Application Publication
(10) Pub. No.: US 2009/0155853 A1
(43) Pub. Date: Jun. 18, 2009

(54) NON-VIRAL GENE DELIVERY COMPLEX

(76) Inventors: Gary Nabel, Washington, DC (US); Wataru Akahata, Kensington, MD (US)

Correspondence Address: KNOBBE, MARTENS, OLSON & BEAR, LLP, 2040 MAIN STREET, FOURTEENTH FLOOR, IRVINE, CA, 92614, US

(21) Appl. No.: 12/092,811

(22) PCT Filed: Nov. 20, 2006
(86) PCT No.: PCT/US06/44525

§ 371 Date: Feb. 24, 2009

Related U.S. Application Data
(60) Provisional application No. 60/737,896, filed on Nov. 18, 2005.
(60) Provisional application No. 60/795,529, filed on Apr. 26, 2006.

(51) Int. Cl. C12P 19/34; C12N 15/74; C12N 15/09; C07H 21/00

(52) U.S. Cl. 435/91.1; 435/320.1; 536/23.1; 435/455

(57) ABSTRACT

The invention relates to fusion proteins useful in delivering a targeted nucleic acid to a target cell, comprising a gene delivery fusion protein (GDFP), said GDFP comprising a nucleic acid binding domain (NBD) that binds to the targeted nucleic acid, fused to a gene delivery domain (GDD) that mediates delivery of the targeted nucleic acid to the target cell, wherein said GDD comprises one or more components that facilitate delivery of a targeted nucleic acid to a target cell, and wherein one of said components is a transport/localization component and wherein said transport/localization component is an adenovirus protein V or derivative thereof that retains protein V activity, and related methods of making and using thereof.

### RELATED APPLICATIONS

This application claims the benefit of U.S. Provisional Application No. 60/737,896 filed Nov. 18, 2005 and U.S. Provisional Application No. 60/795,529 filed Apr. 26, 2006, both of which are hereby incorporated by reference in their entireties.

### FIELD OF THE INVENTION

The field of the invention is gene delivery.

### DESCRIPTION OF THE RELATED ART

Adenovirus type 5 infection is mediated by the interaction of its Fiber with cellular receptors, coxsackie virus B adenovirus receptor (CAR), followed by endocytosis through the interaction between the penton base and the αV integrin on the cell membrane (Meier, O. and Greber, U. F 2003 J Gene Med 5:451-462). The adenovirus is delivered to a slightly acidic intracellular compartment and subsequently escapes to the cytosol by breaking through the endosomal barrier. Once this step occurs, the viral core component is transported through microtubular transport, then docks to the nuclear pore complex where it disassembles, and the genomic DNA is transported to the nucleus.

### Segue to the Invention

We developed various chimeric proteins to use in synthetic vectors. Fiber, penton base and core protein V were fused to DNA binding domains. By utilizing the adenoviral proteins that are implicated in attachment, entry, internalization, escaping from endosomal barrier and genomic DNA transportation, we sought to increase the efficiency of DNA uptake, expression in a variety of cell types, and immunogenicity.

### SUMMARY OF THE INVENTION

The invention relates to fusion proteins useful in delivering a targeted nucleic acid to a target cell, comprising a gene delivery fusion protein (GDFP), said GDFP comprising a nucleic acid binding domain (NBD) that binds to the targeted nucleic acid, fused to a gene delivery domain (GDD) that mediates delivery of the targeted nucleic acid to the target cell, wherein said GDD comprises one or more components that facilitate delivery of a targeted nucleic acid to a target cell, and wherein one of said components is a transport/localization component and wherein said transport/localization component is an adenovirus protein V or derivative thereof that retains protein V activity, and related methods of making and using thereof.

The invention further relates to fusion proteins useful in delivering a targeted nucleic acid to a target cell, comprising a gene delivery fusion protein (GDFP), said GDFP comprising a nucleic acid binding domain (NBD) that binds to the targeted nucleic acid, fused to a gene delivery domain (GDD) that mediates delivery of the targeted nucleic acid to the target cell, wherein said GDD comprises one or more components that facilitate delivery of a targeted nucleic acid to a target cell, and wherein one of said components is a membrane-disrupting component and wherein said membrane-disrupting component is an adenovirus penton base or derivative thereof that retains penton base activity, and related methods of making and using thereof.

The invention also relates to fusion proteins useful in delivering a targeted nucleic acid to a target cell, comprising a gene delivery fusion protein (GDFP), said GDFP comprising a nucleic acid binding domain (NBD) that binds to the targeted nucleic acid, fused to a gene delivery domain (GDD) that mediates delivery of the targeted nucleic acid to the target cell, wherein said GDD comprises one or more components that facilitate delivery of a targeted nucleic acid to a target cell, and wherein one of said components is a binding/targeting component and wherein said binding/targeting component is an adenovirus fiber protein or derivative thereof that retains fiber protein activity, and related methods of making and using thereof.

### BRIEF DESCRIPTION OF THE DRAWINGS

FIG. 1. Schematic representation of Ad5.

FIG. 2. Map and sequence for CMVR-HMGB1-Fiber(car+) (SEQ ID NO: 1).

FIG. 3. Map and sequence for CMVR-His-NLS-HMG-Penton base (SEQ ID NO: 2).

FIG. 4. CMV/R Fiber-His (SEQ ID NO: 3).

FIG. 5. Map and sequence for CMVR-HMG-V (SEQ ID NO: 4).

FIG. 6. Adenovirus 5 Protein V amino acid (SEQ ID NO: 5) and nucleotide (SEQ ID NO: 6) sequences.

FIG. 7. HMG box A amino acid (SEQ ID NO: 7) and nucleotide (SEQ ID NO: 8) sequences.

FIG. 8. HMG-V amino acid sequence (SEQ ID NO: 9).

FIG. 9. Plasmid delivery by chimeric adenovirus 5 Fiber vectors.

FIG. 10. Plasmid delivery by chimeric adenovirus 5 Fiber and Penton Base vectors.

FIG. 11. Plasmid delivery by chimeric adenovirus 5 V vector.

FIG. 12. Chimeric HMG-V/plasmid HIV-1 Env plasmid complex injected into mice.

### DETAILED DESCRIPTION OF THE PREFERRED EMBODIMENT

While vaccination with naked DNA vaccines has shown promise for a variety of disease targets when tested in vivo in small animal models, it has proven to be less efficacious at inducing immune responses in non-human primate models and in human trials. In this study, we developed alternative approaches to delivery of DNA vaccines by creating novel DNA-protein complexes. Targeting of DNA was achieved by making chimeric proteins utilizing specific adenoviral proteins fused to DNA binding proteins that allowed binding to DNA and hence the formation of novel synthetic vectors. Adenoviruses are known to mediate entry into many different cell types, including antigen presenting cells (APC), and transport their genome efficiently. DNA binding domains were fused to adenoviral proteins. The plasmid mixed with these chimeric adenoviral proteins delivered specific genes to target cells efficiently.

### Definitions

Unless defined otherwise, technical and scientific terms used herein have the same meaning as commonly understood by one of ordinary skill in the art to which this invention belongs. See, e.g., Singleton P and Sainsbury D., Dictionary of Microbiology and Molecular Biology 3^{rd} ed., J. Wiley & Sons, Chichester, N.Y., 2001, and Fields Virology 4^{th} ed., Knipe D. M. and Howley P. M. eds, Lippincott Williams & Wilkins, Philadelphia 2001.

The transitional term “comprising” is synonymous with “including,” “containing,” or “characterized by,” is inclusive or open-ended and does not exclude additional, unrecited elements or method steps.

The transitional phrase “consisting of” excludes any element, step, or ingredient not specified in the claim, but does not exclude additional components or steps that are unrelated to the invention such as impurities ordinarily associated therewith.

The transitional phrase “consisting essentially of” limits the scope of a claim to the specified materials or steps and those that do not materially affect the basic and novel characteristic(s) of the claimed invention.

The terms “polypeptide”, “peptide” and “protein” are used interchangeably to refer to polymers of amino acids and do not refer to any particular lengths of the polymers. These terms also include post-translationally modified proteins, for example, glycosylated, acetylated, phosphorylated proteins and the like. Also included within the definition are, for example, proteins containing one or more analogs of an amino acid (including, for example, unnatural amino acids, etc.), proteins with substituted linkages, as well as other modifications known in the art, both naturally occurring and non-naturally occurring.

“Native” polypeptides or polynucleotides refer to polypeptides or polynucleotides recovered from a source occurring in nature. Thus, the phrase “native viral binding proteins” would refer to naturally occurring viral binding proteins.

“Mutein” forms of a protein or polypeptide are those which have minor alterations in amino acid sequence caused, for example, by site-specific mutagenesis or other manipulations; by errors in transcription or translation; or which are prepared synthetically by rational design. Minor alterations are those which result in amino acid sequences wherein the biological activity of the polypeptide is retained and/or wherein the mutein polypeptide has at least 90% homology with the native form.

An “analog” of a polypeptide X includes fragments and muteins of polypeptide X that retain a particular biological activity; as well as polypeptide X that has been incorporated into a larger molecule (other than a molecule within which it is normally found); as well as synthetic analogs that have been prepared by rational design. For example, an analog of a DNA binding protein might refer to a portion of a native DNA binding protein that retains the ability to bind to DNA, to a mutein thereof, to an entire native binding protein that has been incorporated into a recombinant fusion protein, or to an analog of a native binding protein that has been synthetically prepared by rational design.

“Polynucleotide” refers to a polymeric form of nucleotides of any length, either ribonucleotides or deoxyribonucleotides, or analogs thereof. This term refers only to the primary structure of the molecule. Thus, double- and single-stranded DNA, as well as double- and single-stranded RNA are included. It also includes modified polynucleotides such as methylated or capped polynucleotides.

An “analog” of DNA, RNA or a polynucleotide, refers to a macromolecule resembling naturally-occurring polynucleotides in form and/or function (particularly in the ability to engage in sequence-specific hydrogen bonding to base pairs on a complementary polynucleotide sequence) but which differs from DNA or RNA in, for example, the possession of an unusual or non-natural base or an altered backbone. A large variety of such molecules have been described for use in antisense technology.

“Recombinant,” as applied to a polynucleotide, means that the polynucleotide is the product of various combinations of cloning, restriction and/or ligation steps resulting in a construct that is distinct from a polynucleotide found in nature. “Recombinant” may also be used to refer to the protein product of a recombinant polynucleotide. Typically, DNA sequences encoding the structural coding sequence for, e.g., components of the NBD and GDD, can be assembled from cDNA fragments and short oligonucleotide linkers, or from a series of oligonucleotides, to provide a synthetic gene which is capable of being expressed when operably linked to a transcriptional regulatory region. Such sequences are preferably provided in the form of an open reading frame uninterrupted by internal non-translated sequences (i.e., “introns”), such as those commonly found in eukaryotic genes. Such sequences, and all of the sequences referred to in the context of the present invention, can also be generally obtained by PCR amplification using viral, prokaryotic or eukaryotic DNA or RNA templates in conjunction with appropriate PCR amplimers.

A “recombinant expression vector” refers to a polynucleotide which contains a transcriptional regulatory region and coding sequences necessary for the expression of an RNA molecule and/or protein and which is capable of being introduced into a target cell (by, e.g., viral infection, transfection, electroporation or by the non-viral gene delivery (NVGD) techniques of the present invention). A further example would be an expression vector used to express a GDFP of the present invention.

“Recombinant host cells”, “host cells”, “cells”, “target cells”, “cell lines”, “cell cultures”, and other such terms denote higher eukaryotic cells, most preferably mammalian cells, which can be, or have been, used as recipients for recombinant vectors or other transfer polynucleotides, and include the progeny of the original cell which has been transduced. It is understood that the progeny of a single cell may not necessarily be completely identical (in morphology or in genomic or total DNA complement) to the original parent cell, due to natural, accidental, or deliberate mutation.

An “open reading frame” (or “ORF”) is a region of a polynucleotide sequence that can encode a polypeptide or a portion of a polypeptide (i.e., the region may represent a portion of a protein coding sequence or an entire protein coding sequence).

“Fused” or “fusion” refers to the joining together of two or more elements, components, etc., by whatever means (including, for example, a “fusion protein” made by chemical conjugation (whether covalent or non-covalent), as well as the use of an in-frame fusion to generate a “fusion protein” by recombinant means, as discussed infra. An “in-frame fusion” refers to the joining of two or more open reading frames (ORFs), by recombinant means, to form a single larger ORF, in a manner that maintains the correct reading frame of the original ORFs. Thus, the resulting recombinant fusion protein is a single protein containing two or more segments that correspond to polypeptides encoded by the original ORFs (which segments are not normally so joined in nature). Although the reading frame is thus made continuous throughout the fused segments, the segments may be physically separated by, for example, in-frame flexible polypeptide linker sequences (“flexons”), as described infra.

A “flexon” refers to a flexible polypeptide linker sequence (or to a nucleic acid sequence encoding such a polypeptide) which typically comprises amino acids having small side chains (e.g., glycine, alanine, valine, leucine, isoleucine and serine). In the present invention, flexons can be incorporated in the GDFP between one or more of the various domains and components. Incorporating flexons between these components is believed to promote functionality by allowing them to adopt conformations relatively, independently from each other. Most of the amino acids incorporated into the flexon will preferably be amino acids having small side chains. The flexon will preferably comprise between about four and one hundred amino acids, more preferably between about eight and fifty amino acids, and most preferably between about ten and thirty amino acids.

A “transcriptional regulatory region” or “transcriptional control region” refers to a polynucleotide encompassing all of the cis-acting sequences necessary for transcription, and may include sequences necessary for regulation. Thus, a transcriptional regulatory region includes at least a promoter sequence, and may also include other regulatory sequences such as enhancers, transcription factor binding sites, polyadenylation signals and splicing signals.

“Operably linked” refers to a juxtaposition wherein the components so described are in a relationship permitting them to function in their intended manner. For instance, a promoter sequence is operably linked to a coding sequence if the promoter sequence promotes transcription of the coding sequence.

“Transduction,” as used herein, refers to the introduction of an exogenous polynucleotide into a host cell, irrespective of the method used for the insertion, which methods include, for example, transfection, viral infection, transformation, electroporation and the non-viral gene delivery techniques of the present invention. The introduced polynucleotide may be stably or transiently maintain d in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome.

A “sequence-specific nucleic acid binding protein” is a protein that binds to nucleic acids in a sequence-specific manner, i.e., a protein that binds to certain nucleic acid sequences (i.e., “cognate recognition sequences”, infra) with greater affinity than to other nucleic acid sequences. A “sequence-non-specific nucleic acid binding protein” is a protein that binds to nucleic acids in a sequence-non-specific manner, i.e., a protein that binds generally to nucleic acids.

A “cognate” receptor of a given ligand refers to the receptor normally capable of binding such a ligand. A “cognate” recognition sequence is defined as a nucleotide sequence to which a nucleic acid binding domain of a sequence-specific nucleic acid binding protein binds with greater affinity than to other nucleic acid sequences. A “cognate” interaction refers to an intermolecular association based on such types of binding (e.g., an association between a receptor and its cognate ligand, and an association between a sequence-specific nucleic acid binding protein and its cognate nucleic acid sequence).

“Gene delivery” is defined as the introduction of targeted nucleic acid into a target cell for gene transfer and may encompass targeting/binding, uptake and transport/localization.

### Adenoviruses

Fifty one human adenovirus serotypes (Table 1) have been distinguished on the basis of their resistance to neutralization by antisera to other known adenovirus serotypes. Type-specific neutralization results predominantly from antibody binding to epitopes on the virion hexon protein and the terminal knob portion of the Fiber protein. Hypervariable regions have been identified on the hexon that make up serotype-specific loops on the surface of the protein. The various serotypes are classified into six subgroups (see Table 1) based on their ability to agglutinate red blood cells. The central shaft of the viral Fiber protein is responsible for binding to erythrocytes, and the hemagglutination reaction of adenovirus is inhibited by antisera specific for viruses of the same type but not by antisera to viruses of different types. Most of the structural studies of adenoviruses have focused on the closely related adenoviruses type 2 and 5 (Ad2 and Ad5).

TABLE 1
Human Adenoviruses

| Subgroup | Hemagglutination Groups | Serotypes |
|---|---|---|
| A | IV (little or no | 12, 18, 31 |
| | agglutination) | |
| B | I (complete agglutination | 3, 7, 11, 14, 16, 21, 34, 35, 50 |
| | of monkey erythrocytes) | |
| C | III (partial agglutination | 1, 2, 5, 6 |
| | of rat erythrocytes) | |
| D | II (complete agglutination | 8, 9, 10, 13, 15, 17, 19, 20, |
| | of rat erythrocytes) | 22-30, 32, 33, 36-39, 42-49, 51 |
| E | III | 4 |
| F | III | 40, 41 |

Genbank accession numbers that contain representative amino acid and nucleotide sequences for the human adenovirus subgroups and serotypes are listed in Tables 2 and 3, respectively.

TABLE 2
Representative Genbank Accession Numbers
for Human Adenovirus Subgroups.

| Subgroup | Genbank Accession Number |
|---|---|
| Human adenovirus A | NC_001460 |
| Human adenovirus B | NC_004001 |
| Human adenovirus C | NC_001405 |
| Human adenovirus D | NC_002067 |
| Human adenovirus E | NC_003266 |
| Human adenovirus F | NC_001454 |

TABLE 3
Representative Genbank Accession Numbers
for Human Adenovirus Serotypes.

| Serotype | Subtype | Genbank Accession Number |
|---|---|---|
| Type 1 | Human Adenovirus C | AC_000017 |
| Type 2 | Human Adenovirus C | AC_000007 |
| Type 3 | Human Adenovirus B | |
| Type 4 | Human Adenovirus E | |
| Type 5 | Human Adenovirus C | AC_000008 |
| Type 6 | Human Adenovirus C | |
| Type 7 | Human Adenovirus B | AC_000018 |
| Type 8 | | |
| Type 9 | | |
| Type 10 | Human Adenovirus D | |
| Type 11 | Human Adenovirus B | AC_000015 |
| Type 12 | Human Adenovirus A | AC_000005 |
| Type 13 | Human Adenovirus D | |
| Type 14 | Human Adenovirus B | |
| Type 15 | Human Adenovirus D | |
| Type 16 | Human Adenovirus B | |
| Type 17 | Human Adenovirus D | AC_000006 |
| Type 18 | Human Adenovirus A | |
| Type 19 | Human Adenovirus D | |
| Type 20 | Human Adenovirus D | |
| Type 21 | Human Adenovirus B | |
| Type 22 | Human Adenovirus D | |
| Type 23 | Human Adenovirus D | |
| Type 24 | Human Adenovirus D | |
| Type 25 | Human Adenovirus D | |
| Type 26 | Human Adenovirus D | |
| Type 27 | Human Adenovirus D | |
| Type 28 | Human Adenovirus D | |
| Type 29 | Human Adenovirus D | |
| Type 30 | Human Adenovirus D | |
| Type 31 | Human Adenovirus A | |
| Type 32 | Human Adenovirus D | |
| Type 33 | Human Adenovirus D | |
| Type 34 | Human Adenovirus B | |
| Type 35 | Human Adenovirus B | AC_000019 |
| Type 36 | Human Adenovirus D | |
| Type 37 | Human Adenovirus D | |
| Type 38 | Human Adenovirus D | |
| Type 39 | Human Adenovirus D | |
| Type 40 | Human Adenovirus F | |
| Type 41 | Human Adenovirus F | |
| Type 42 | Human Adenovirus D | |
| Type 43 | Human Adenovirus D | |
| Type 44 | Human Adenovirus D | |
| Type 45 | Human Adenovirus D | |
| Type 46 | Human Adenovirus D | |
| Type 47 | Human Adenovirus D | |
| Type 48 | Human Adenovirus D | |
| Type 49 | Human Adenovirus D | DQ393829 |
| Type 50 | Human Adenovirus B | |
| Type 51 | Human Adenovirus D | |

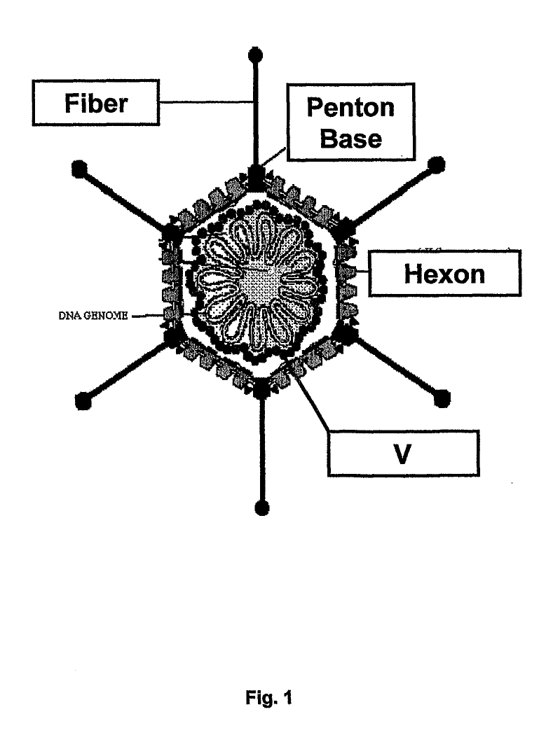

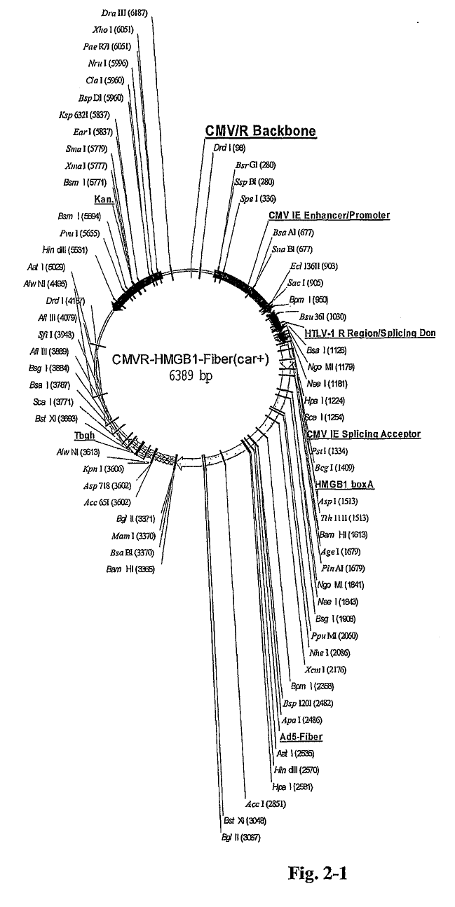

Referring to FIG. 1, Adenoviruses (Ads) are nonenveloped virions 70-90 nm in diameter with a capsid consisting of three main exposed structural proteins, the hexon, fiber, and penton base. Hexon accounts for the majority of the structural components of the capsid, which consists of 240 trimeric hexon capsomeres and 12 pentameric penton bases. Protein V can bind to a penton base and it might bridge between the core and capsid, positioning one relative to the other. The trimeric fiber protein protrudes from the penton base at each of the 12 vertices of the capsid and is a knobbed rod-like structure. The most remarkable and obvious difference in the surface of adenovirus capsids compared to that of most other icosahedral viruses is the presence of the long, thin fiber protein (FIG. 1). The primary role of the fiber protein is the tethering of the viral capsid to the cell surface via its interaction with a cellular receptor. The fiber protein is exquisitely adapted for such a purpose. The fiber proteins of all human adenovirus serotypes share a common architecture: an N-terminal tail, a central shaft made of repeating sequences, and a C-terminal globular knob domain (FIG. 1). The first approximately 45 residues of the fiber are highly conserved among different serotypes and are responsible for binding to the penton base.

The recombinant proteins of the present invention can be amplified from any of the available human adenovirus serotypes types 1-51.

### Non-Viral Gene Delivery Complex

As is described in detail below, the non-viral gene delivery complexes of the present invention comprise gene delivery fusion proteins (GDFPs) that bind targeted nucleic acid through a nucleic acid binding domain (NBD) and facilitate gene delivery through a gene delivery domain (GDD). Each of these domains can comprise a number of different functional components and sub-components. Some of these potential components are summarized in the following list:

1. Gene Delivery Fusion Protein (GDFP)
A. Nucleic Acid Binding Domain (NBD)
B. Gene Delivery Domain (GDD)
   (1) Binding/Targeting (B/T) component
   (2) Membrane-Disrupting (M-D) component
   (3) Transport/Localization (T/L) component

2. Targeted Nucleic Acid (tNA)
A. Binding sites for the GDFP
B. Sequence of interest (e.g., gene to be delivered)
C. Other possible sequences (e.g., selectable markers)

Each of these domains and components, as well as additional elements that may be included, are defined and described in detail below.

The practice of the present invention will employ, unless otherwise indicated, a number of conventional techniques of molecular biology, microbiology, recombinant DNA, and immunology, which are within the skill of the art. Such techniques are explained fully in the literature, see, e.g., Sambrook, J., Russell, D. W. (2001) Molecular Cloning: A Laboratory Manual, the third edition, Cold Spring Harbor Laboratory Press, Cold Spring Harbor, N.Y. and F. M. Ausubel et al. (eds.), Current Protocols in Molecular Biology, (1987 and 1993).

### The Gene Delivery Fusion Protein/Targeted Nucleic Acid Complex (GDFP/tNA)

One concept of the present invention is to create recombinant gene delivery fusion proteins (GDFPs) that are non-sequence-specific in their binding to nucleic acid that facilitate delivery of the tNA into a target cell. The GDFPs bind targeted nucleic acid through a nucleic acid binding domain (NBD) and facilitate gene delivery through a gene delivery domain (GDD).

Thus the context of the present invention, targeted nucleic acids can be delivered via one or more steps that are mediated or augmented by GDFPs. In particular, the gene delivery process can include one or more of the following steps: (1) binding and/or targeting of the GDFP/tNA complex to the surface of a target cell, (2) uptake of the tNA (with or without the GDFP) by the target cell, and (3) intracellular transport and/or localization of the tNA to an organelle such as the nucleus. The individual domains and components of the GDFP/tNA complex and their construction and assembly are described in more detail below.

### 1. The Gene Delivery Fusion Protein (GDFP)

The GDFP comprises two major domains, a nucleic acid binding domain (NBD) and a gene delivery domain (GDD). Each of these major domains comprises one or more components facilitating nucleic acid binding and gene delivery, respectively. These individual components may be derived from naturally-occurring proteins, or they may be synthetic (e.g., an analog of a naturally-occurring component). Typically, cloned DNA encoding various components will already be available as plasmids although it is also possible to synthesize polynucleotides encoding the components based upon published sequence information. Polynucleotides encoding the components can also be readily obtained using polymerase chain reaction (PCR) methodology.

In the construction of the GDFP, discussed in more detail below, DNA sequences encoding the domains and their various components are preferably fused in-frame so that the GDFP can be conveniently synthesized as a single polypeptide chain (i.e., not requiring further assembly). The various domains and components can also be separated by flexible peptide linker sequences called “flexons” which are defined in more detail above.

### A. The Nucleic Acid Binding Domain (NBD)

A nucleic acid binding domain is a length of polypeptide capable of binding (either directly or indirectly) to the targeted nucleic acid (tNA) with an affinity adequate to allow the gene delivery domain of the GDFP to mediate or augment the delivery of the tNA into a target cell. Most conveniently, the NBD will bind directly to the tNA without the need for any intermediary binding element.

In sequence-specific GDFPs, the NBD contains a sequence-specific binding component that is an analog of a sequence-specific nucleic acid binding protein. In one embodiment of this type, the component allows the nucleic acid binding by the NBD to be sequence-specific with respect to the tNA, in which case the NBD may bind to a specific cognate recognition sequence within the tNA.

The NBD may comprise, for example, a known nucleic acid binding protein, or a nucleic acid binding region thereof. The NBD may also comprise two or more nucleic acid binding regions derived from the same or different nucleic acid binding proteins. Such multimerization of nucleic acid binding regions in the NBD can allow for the interaction of the GDFP with the targeted nucleic acid to be of desirable specificity and/or higher affinity. This strategy can be used alone or in combination with multimerization of recognition sequence motifs in the tNA to increase binding avidity, as discussed below.

DNA encoding the NBD domain of the GDFP may be obtained from many different sources. For example, many proteins that are capable of binding nucleic acid have been molecularly cloned and their cognate target recognition sequences have been identified. Such sequence-specific binding proteins include, for example, regulatory proteins such as those involved in transcription or nucleic acid replication, and typically have a modular construction, consisting of distinct DNA binding domains and regulatory domains. A number of families of such nucleic acid binding proteins have been characterized on the basis of recurring structural motifs including, for example, Helix-Turn-Helix proteins such as the bacteriophage lambda cI repressor; homeodomain proteins such as the Drosophila Antennapedia regulator; the POU domain present in proteins such as the mammalian transcription factor Oct2; Zinc finger proteins (e.g., GAL4); steroid receptors; leucine zipper proteins (e.g., GCN4, C/EBP and c-jun); beta-sheet motifs (e.g., the prokaryotic Arc repressor); and other families (including serum response factor, oncogenes such as c-myb, NFκB, RelA and others).

For many of these proteins, the nucleic acid binding domains have been mapped in detail; and, for a number of such domains, recombinant fusions with heterologous sequences have been made and shown to retain the binding activities of the parental DNA binding domain. For example, in the case of the yeast-derived transcriptional activator GAL4, the DNA binding domain has been defined, and fusions of this domain to heterologous adjoining sequences have been made that retain DNA sequence-specific binding activity. This ability to functionally “swap” binding domains has also been shown for a number of other DNA binding proteins, including, for example, the E. coli lexA repressor, the yeast transcriptional activator GCN4, the bacteriophage lambda cI repressor, the mammalian transcription factors Sp1 and C/EBP. Similarly, functional swapping has been reported in the nuclear DNA-binding steroid hormone receptors. Sequence-specific nucleic acid binding proteins can exhibit a range of binding affinities to different cognate nucleic acid sequences in vitro.

Virally encoded nucleic acid binding proteins can also be used in the present invention. These include, for example, the adenovirus E2A gene product, which can bind single-stranded DNA, double-stranded DNA and also RNA, the retroviral IN proteins, the AAV rep 68 and 78 proteins and the SV40 T antigen. The cellular p53 gene product, which binds T antigen, is also a DNA binding protein.

Similarly, RNA binding proteins have been identified and their inclusion in the NBD would associate the GDFP with a targeted RNA and thereby achieve RNA delivery mediated by the gene delivery domain of the GDFP. RNA binding proteins that can be used in the context of the present invention include, for example, the Tat and Rev proteins of HIV. Similarly, cellular RNA binding proteins, such as the interferon-inducible 9-27 gene product can also be used.

Non-sequence-specific binding proteins include, for example, histones, proteins such as nucleolin, polybasic polypeptide sequences such as polylysine or polyarginine, the non-histone high mobility group proteins (e.g., HMGB-1 box A), polycationic amphipathic polypeptides such as LAH4 protein, protamine, and other proteins that interact non-specifically with nucleic acids.

### B. The Gene Delivery-Domain (GDD)

The GDD portion of the GDFP contains one or more polypeptide regions that mediate or augment the efficiency of gene delivery. Such sequences may include, for example, binding/targeting components, membrane-disrupting components, or transport/localization components.

A particular GDD need not contain a component representing each of the aforementioned types. Conversely, a GDD may contain more than a single component of a given type to obtain the desired activity. Moreover, a particular segment of a GDD might serve the function of two or more of these components. For example, a single region of a polypeptide might function both in binding to a cell surface and in disrupting of the cell membrane.

### (1) Binding/Targeting (B/T) Components

Binding/targeting components are regions of polypeptides that mediate binding to cellular surfaces (which binding may be specific or non-specific, direct or indirect). Any protein that can bind to the surface of the desired target cell can be employed as a source of B/T components. Such proteins include, for example, ligands that bind to particular cell surface receptors, antibodies, lectins, cellular adhesion molecules, viral binding proteins and any other proteins that associate with cellular surfaces. The “receptors” for these binding proteins include but are not limited to proteins. Moreover, the receptors may, but need not, be specific and/or restricted to certain cell types. Essentially, the B/T components can be prepared from any ligand that binds to a cell surface molecule.

The ligands suitable for targeting a particular sub-population of cells will be those which bind to receptors present on cells of that sub-population. Taking cytokines as an example, the target cells for a large number of these molecules are already known, and, in many cases, the particular cell surface receptors for the cytokine have already been identified and characterized. Typically, the cell surface receptors for cytokines are transmembrane glycoproteins that consist of either a single chain polypeptide or multiple protein subunits. The receptors generally bind to their cognate ligands with high affinity and specificity, and may be widely distributed on a variety of somatic cells, or quite specific to given cell subsets. The presence of cytokine receptors on a given cell type can also be predicted from the ability of a cytokine to modulate the growth or other characteristics of the given cell; and can be determined, for example, by monitoring the binding of a labeled cytokine to such cells.

The choice of a particular ligand will depend on the presence of cognate receptors on the desired target cells. It may also depend on the corresponding absence of cognate receptors on other cells which it may be preferable to avoid targeting. With the cytokines, for example, the role of particular molecules in the regulation of various cellular systems is well known in the art. In the hematopoietic system, for example, the hematopoietic colony-stimulating factors and interleukins regulate the production and function of mature blood-forming cells. Lymphocytes are dependent upon a number of cytokines for proliferation.

The choice of a particular ligand may also be influenced by other activities that may be possessed by the ligand (besides binding to the cell surface). The rapidity with which novel ligands and their cognate receptors have recently been molecularly cloned has generated a wide array of these molecules. In particular, the combination of direct cDNA expression cloning and screening assays for either induction of proliferation of binding to specific cell surface receptors on target cells has led to many new molecules being cloned. The advent of these technologies will undoubtedly lead to the cloning of more ligands, including cytokines and other proteins.

While the foregoing principles have been illustrated using cytokines as a convenient example, these principles are also applicable to other ligands capable of binding to cell surfaces, including for example, antibodies, lectins, cellular adhesion molecules, viral binding proteins and any other proteins that associate with cellular surfaces.

Proteins capable of targeting the GDD and thus the GDFP/tNA complex to cell surfaces can be derived from viruses. Many such viral proteins capable of binding to cells have been identified, including, for example, the well-known envelope (“env”) proteins of retroviruses; hemagglutinin proteins of RNA viruses such as the influenza virus; spike proteins of viruses such as the Semliki Forest virus and proteins from non-enveloped viruses such as adenoviruses (see, e.g., Wickham et al. 1993 Cell 73:309-319).

By way of illustration, the B/T components of the present invention can thus be derived from a portion of a viral binding protein that is normally involved in mediating binding or targeting of the virus into a host cell, or a mutein of such a portion of a binding or targeting protein. The portion of the GDFP that may be derived from such a viral binding or targeting protein may, but need not, also contain the portion of the binding protein that causes membrane disruption as described below.

### 2. Membrane-Disrupting (M-D) Components

Membrane-disrupting components are protein sequences capable of locally disrupting cellular membranes such that the GDFP/tNA complex can traverse a cellular membrane. M-D components facilitating uptake of the GDFP-targeted nucleic acid complex by target cells are typically membrane-active regions of protein structure having a hydrophobic character. Such regions are typical in membrane-active proteins involved in facilitating cellular entry of proteins or particles.

For example, viruses commonly enter cells by endocytosis and have evolved mechanisms for disrupting endosomal membranes. Many viruses encode surface proteins capable of disrupting cellular membranes including, for example, retroviruses, influenza virus, Sindbis virus, Semliki Forest virus, Vesicular Stomatitis virus, Sendai virus, vaccinia virus, and adenovirus. The mechanism for viral entry, in which a viral binding protein binds to a specific cell surface receptor and subsequently mediates virus entry, frequently by means of a hydrophobic membrane-disruptive domain, is a common theme among viruses, including adenovirus, and many such molecules are known to those skilled in the art.

By way of illustration, the M-D components of the present invention can thus be derived from a portion of a viral binding protein that is normally involved in mediating uptake of the virus into a host cell, or a mutein of such a portion of a binding protein. The portion of the GDFP that may be derived from such a viral binding protein may, but need not, also contain the portion of the binding protein that causes the viral particle to associate with a specific receptor on a target cell (which latter portion may thus function as a B/T component, as described above).

### (3) Transport/Localization (T/L) Components

Transport/localization components mediate or augment the transport and/or localization of the GDFP/tNA complex to a particular sub-cellular compartment such as the nucleus.

A number of sequences that mediate transport and/or localization of proteins have been identified. These include, by way of illustration, the adenovirus 5 protein V, a basic, arginine-rich protein. Other examples include the nuclear localization sequence (nls) of, for example, SV40 T antigen and the HIV matrix protein. These are typically short basic peptide sequences, and may also be bipartite basic sequences. Nuclear localization sequences have been fused to heterologous proteins and shown to confer on them the property of nuclear localization. These sequences can be readily incorporated into the GDD by recombinant DNA methodology to facilitate nuclear localization of the desired GDFP/tNA complex.

By way of illustration, the T/L components of the present invention can thus be derived from a portion of a viral protein that is involved in mediating transport or localization, or a mutein of such a portion of a transport/localization protein.

### Targeted Nucleic Acids (tNA)

The targeted nucleic acid (tNA) is a polynucleotide, or analog thereof, to be delivered to a target cell. Thus, targeted nucleic acids include, for example, oligonucleotides and longer polymers of DNA, RNA or analogs thereof, in double-stranded or single-stranded form. The tNA may be circular, supercoiled or linear. A preferred example of a targeted nucleic acid is a DNA expression vector comprising a gene (or genes) of interest operably linked to a transcriptional control region (or regions). The transcriptional control region may be selected so as to be specifically activated in the desired target cells, or to be responsive to specific cellular or other stimuli.

Targeted nucleic acids may also include, for example, positive and/or negative selectable markers; thereby allowing the selection for and/or against cells stably expressing the selectable marker, either in vitro or in vivo.

Use of the present invention to deliver RNA would enable the introduction of RNA decoys, ribozymes and antisense nucleic acids, for example.

In sequence-specific GDFPs, the targeted nucleic acids are recognized and bound by the GDFP by virtue of specific cognate recognition sequences to which the nucleic acid binding domain (NBD) of the sequence-specific GDFP binds. Both DNA and RNA binding domains have been isolated from proteins that bind to particular nucleic acids in a sequence-specific fashion. Inclusion of such a cognate recognition sequence in the targeted nucleic acid allows for specific binding of the GDFP to the tNA. Recognition sites for many nucleic acid binding proteins have been identified.

Binding of sequence-specific binding proteins to DNA tends to be more avid when the recognition sequence motif is multimerized. Accordingly, the cognate recognition sequences may be multimerized in the targeted nucleic acids so as to enhance the binding affinity or selectivity of a GDFP for its cognate tNA. This could also have other advantages, such as increasing the effective amount of the GDFP bound to the tNA, or promoting compaction/condensation of the tNA by sequence-specific or sequence-non-specific NBD components.

Typically, but not necessarily, the cognate recognition sequences in expression vectors will be placed in the plasmid backbone of the vector. This also applies to other cis-acting sequences that are needed in the tNA to facilitate gene delivery. However, it may be desirable to remove plasmid backbone sequences from the DNA to be transferred. In this case, the expression cassette can be conveniently flanked by restriction enzyme sites, such that restriction enzyme digestion separates the backbone from the mammalian expression cassette. The expression cassette can then be purified away from the plasmid backbone for use in transduction experiments. In this case the cognate recognition sequence (CRS) would be located on the fragment bearing the expression cassette. It is also possible to construct the GDFP so as to bind to more than one tNA.

As discussed above, the tNA can also be bound to the GDFP via sequence-nonspecific interactions in addition to sequence-specific interactions. In a sequence-specific GDFP, such sequence-non-specific interactions can be mediated by auxiliary components derived from sequence-non-specific binding proteins, as discussed above. Such auxiliary non-specific binding components can also serve to compact or otherwise reconfigure the targeted nucleic acid.

### Assembly of GDFPs

Preferably, the GDFP is prepared as a single polypeptide fusion protein generated by recombinant DNA methodology. To generate such a GDFP, sequences encoding the desired components of the GDFP are assembled and fragments ligated into an expression vector. Sequences encoding the various components may be assembled from other vectors encoding the desired protein sequence, from PCR-generated fragments using cellular or viral nucleic acid as template nucleic acid, or by assembly of synthetic oligonucleotides encoding the desired sequence. However, all nucleic acid sequences encoding such a preferred GDFP should preferably be assembled by in-frame fusions of coding sequences. Flexons, described above, can be included between various components and domains in order to enhance the ability of the individual components to adopt configurations relatively independently of each other.

Although a sequence-specific GDFP is preferably assembled and expressed as a single polypeptide chain, one or more of its domains or components may be produced as a separate chain that is subsequently linked to the GDFP by, e.g., disulfide bonds, or chemical conjugation. It is also feasible to prepare complexes in which domains such as the NBD and the GDD or their components are physically associated by other than recombinant means, either directly or indirectly, for example, by virtue of non-covalent interactions, or via co-localization on a proteinaceous or lipid surface.

The GDFP may be expressed either in vitro, or in a prokaryotic or eukaryotic host cell, and can be purified to the extent necessary. An alternative to the expression of GDFPs in a host cell is synthesis in vitro. This may be advantageous in circumstances in which high levels of expression of a GDFP might interfere with the host cell's metabolism; and can be accomplished using any of a variety of cell-free transcription/translation systems that are known in the art. GDFPs can also be prepared synthetically. It will likely be desirable for the GDFP to possess a component or sequence that can facilitate the detection and/or purification of the GDFP. Such a component may be the same as or different from one of the various components described above.

Many approaches of expressing and purifying recombinant proteins are known to those skilled in the art, and kits for recombinant protein expression and purification are available from several commercial manufacturers of molecular biology products. Typically, an increased level of purity of the GDFP will be desirable. However, because of the specificity of the GDFP for nucleic acid binding, the degree of purification need not necessarily be extensive. The GDFPs of the present invention may be sterilized by simple filtration through a 0.22 or 0.45 μm filter so as to avoid microbial contamination of the target cells.

Since the domains of the GDFP can be assembled in modular fashion in an expression vector, its construction by recombinant DNA methodology allows the GDD to consist of one or many components. Such components may have complementing activity in mediating or enhancing gene delivery, or they may have closely related functions. In essence, the gene delivery domain can be viewed as possessing any function that mediates or enhances the efficiency of delivery of the tNA bound to the GDFP.

### Other Variations of GDFPs

Other variations will be apparent to those of skill in the art. For example, the GDFP may itself be multimerized. Multimerization may be advantageous to increase avidity of binding of either the NBD or the GDD. A given tNA molecule may also contain multiple distinct cognate recognition sequences, different sequence-specific GDFPs with distinct functions, or the tNA may be bound with a mixture of sequence-specific and non-sequence-specific GDFPs. Additionally, certain components of the GDD, such as integrase (IN) proteins, may require dimerization for optimal activity. Dimerization of the GDFP may be obtained by including, for example, a leucine zipper motif in the GDFP. Such motifs are common in DNA binding proteins and are responsible for their dimerization. Leucine zippers can be inserted into DNA binding proteins and cause them to dimerize. Multimerization of GDFPs can also be achieved, for example, by creating a recombinant fusion protein that contains two or more GDFPs. Preferably such multimerized GDFPs are separated by flexons, as described herein. Other oligomerization motifs from dimeric or multimeric proteins can similarly be employed.

### Non-Sequence-Specific Fusion Proteins

Non-sequence-specific GDFPs do not bind targeted nucleic acids in a sequence-specific manner because the nucleic acid binding components of the GDFPs are derived from nucleic acid binding proteins that are non-sequence-specific in their binding to nucleic acid.

### Nucleic Acid Binding Domains of Non-Sequence-Specific GDFPs

The nucleic acid binding domains (NBDs) of non-sequence-specific GDFPs comprise binding components that are derived from non-sequence-specific nucleic acid binding proteins, recombinantly fused to a gene delivery domain (GDD) as described above.

A number of non-sequence-specific nucleic acid binding proteins have been identified and characterized, including, for example, histones or polypeptides derived therefrom, retroviral nucleocapsid proteins, proteins such as nucleolin, avidin, and polybasic polypeptide sequences such as polylysine and polyarginine.

For the reasons discussed herein, all of the GDFPs of the present invention are preferably produced as recombinant fusion proteins. However, the recombinant expression, in a host cell, of non-sequence-specific nucleic acid binding components in non-sequence-specific GDFPs (as well as in sequence-specific GDFPs) may be hindered by interference of the expressed proteins with host cell nucleic acids. In such situations, the GDFPs can be readily synthesized in vitro using any of a variety of cell-free transcription/translation systems that are known in the art.

### Gene Delivery Domains of Non-Sequence-Specific GDFPs

The various possible sources of components making up the gene delivery domains of non-sequence-specific GDFPs are essentially the same as described above for sequence-specific GDFPs (although, by definition, non-sequence-specific GDFPs would not include sequence-specific binding components).

### Targeted Nucleic Acids for Use with Non-Sequence-Specific GDFPs

The targeted nucleic acids to be combined with non-sequence-specific GDFPs are as described above except that they need not contain specific recognition sequences since the non-sequence-specific GDFPs bind nucleic acids via non-specific interactions.

### Assembly of Non-Sequence-Specific GDFPs

The assembly of non-sequence-specific GDFPs is preferably via the synthesis of recombinant fusion proteins (see the description above regarding assembly of sequence-specific GDFPs).

### Gene Delivery and Genetic Immunization

The GDFPs of the present invention can be used for in vitro or in vivo gene delivery. For therapeutic applications, target cells can be transduced ex vivo and returned to a patient, or, given the biochemical nature of the tNA/GDFP complex, cells can be treated directly in vivo. For such in vivo therapy, the complexes can be formulated for a variety of modes of administration, including systemic and topical or localized administration. Techniques and formulations may be found, (e.g., in Remington's Pharmaceutical Sciences, 18th edition, Mack Publishing Co., Easton, Pa., 1990). The tNA/GDFP complex may be combined with a carrier such as a diluent or excipient which may include, for example, fillers, extenders, wetting agents, disintegrants, surface-active agents, or lubricants, depending on the nature of the mode of administration and the dosage forms. The nature of the mode of administration will depend, for example, on the location of the desired target cells. For in vivo administration, injection is preferred, including intramuscular, intratumoral, intravenous, intra-arterial (including delivery by use of double balloon catheters), intraperitoneal, and subcutaneous. Delivery to lung tissue can be accomplished by, e.g., aerosolization. For injection, the complexes of the invention are formulated in liquid solutions, preferably in physiologically compatible buffers such as Hank's solution or Ringer's solution. In addition, the complexes may be formulated in solid form and redissolved or suspended immediately prior to use. Lyophilized forms are also included. Systemic administration can also be by transmucosal or transdermal means, or the compounds can be administered orally. For transmucosal or transdermal administration, penetrants appropriate to the barrier to be permeated are used in the formulation. For topical administration, the complexes of the invention may be formulated into ointments, salves, gels or creams, as is generally known in the art.

The GDFP approach can thus be used to target any cell, in vitro, ex vivo or in vivo, the only requirement being that the target cells have binding sites for the GDFP on their surface. The present invention will thus be useful for many gene therapy applications. An illustrative application of the present invention is delivery of DNA or RNA to antigen presenting cells (APCs). This could be useful, for example, to allow expression of specific (tNA-encoded) antigens by an APC, thereby allowing the APC to stimulate an antigen-specific immune response, such as a cytotoxic T lymphocyte (CTL) response. Such an approach can be used in vitro, by transduction of APCs with a GDFP/tNA complex thereby allowing antigen presentation for the stimulation and generation of CTLs in vitro, or in vivo delivery can be used, to allow such antigen presentation in vivo. For example, the HIV envelope (env) plasmid with HMG-V can be delivered to the APCs of subjects. Direct delivery of RNA to APCs using the present invention may be especially desirable for situations in which antigens are encoded by transcripts that require special conditions for intracellular transport or processing that may not happen efficiently in the APC. Transduction of APCs with RNA in the context of the present invention can thus be used, for example, to circumvent the need for nuclear export of rev-dependent RNAs. Additionally, the present invention could be used to introduce genes into hepatocytes of the liver to correct genetic defects such as familial hypercholesterolemia, hemophilia and other metabolic disorders, or to produce recombinant products for systemic delivery.

The non-viral gene delivery complexes of the invention are useful for purposes of genetic vaccination. In such applications, a suitable non-viral gene delivery complex of the invention can be introduced into cells in culture, followed by introduction of the cells subsequently into the subject, i.e., ex vivo administration of the non-viral gene delivery complex. Alternatively, the non-viral gene delivery complex can be introduced into the cells of the subject by administering the non-viral gene delivery complex directly to the subject. The choice of non-viral gene delivery complex and mode of administration will vary depending on the particular application.

The non-viral gene delivery complexes of the invention are useful for treating and/or preventing various diseases and other conditions. The non-viral gene delivery complexes can be delivered to a subject to induce an immune response. Suitable subjects include, but are not limited to, a mammal, including, e.g., a human, primate, monkey, orangutan, baboon, mouse, pig, cow, cat, goat, rabbit, rat, guinea pig, hamster, horse, sheep; or a non-mammalian vertebrate such as a bird (e.g., a chicken or duck) or a fish, or invertebrate.

The dose administered to a patient, in the context of the present invention should be sufficient to affect a beneficial effect, such as an immune or other prophylactic or therapeutic response in the patient over time. The dose will be determined by the efficacy of the particular non-viral gene delivery complex employed and the condition of the patient, as well as the body weight or vascular surface area of the patient to be treated. The size of the dose also will be determined by the existence, nature, and extent of any adverse side-effects that accompany the administration of a particular non-viral gene delivery complex, or transduced cell type in a particular patient.

In determining the effective amount of the non-viral gene delivery complex to be administered in the treatment or prophylaxis of an infection or other condition, the physician evaluates non-viral gene delivery complex toxicities, progression of the disease and possible production of anti-adenovirus antibodies. In general, the dose equivalent of a targeted nucleic acid for a typical 70 kilogram patient can range from about 10 ng to about 1 g, about 100 ng to about 100 mg, about 1 μg to about 10 mg, about 10 μg to about 1 mg, or from about 30 to 300 μg. Doses of non-viral gene delivery complexes are calculated to yield an equivalent amount of targeted nucleic acid. Administration can be accomplished via single or divided doses.

The toxicity and therapeutic efficacy of the non-viral gene delivery complexes provided by the invention are determined using standard pharmaceutical procedures in cell cultures or experimental animals. One can determine the LD50 (the dose lethal to 50% of the population) and the ED50 (the dose therapeutically effective in 50% of the population) using procedures presented herein and those otherwise known to those of skill in the art.

The non-viral gene delivery complexes of the invention can be packaged in packs, dispenser devices, and kits for administering the non-viral gene delivery complexes to a mammal. For example, packs or dispenser devices that contain one or more unit dosage forms are provided. Typically, instructions for administration of the non-viral gene delivery complexes will be provided with the packaging, along with a suitable indication on the label that the non-viral gene delivery complex is suitable for treatment of an indicated condition. For example, the label may state that the active ingredient within the packaging is useful for treating a particular infectious disease or preventing or treating other diseases or conditions that are mediated by, or potentially susceptible to, a mammalian immune response.

### Delivery of DNA into Cells using Targeting with Novel DNA Chimeric Viral Protein Constructs

Alternative DNA vaccine approaches to deliver plasmid efficiently were developed in order to enhance immunogenicity. Chimeric proteins utilizing specific adenoviral proteins, fiber, penton base and core protein V fused to DNA binding proteins were created. The plasmid mixed with the chimeric adenoviral proteins were delivered efficiently to 293T cells and CHO cells expressing coxsackie virus B adenovirus receptor (CAR). In particular, the plasmid with the chimeric core protein V was delivered efficiently to dendritic cells (DC) as well as 293T cells. These vectors provide a novel method to induce DNA vaccine immunogenicity in vivo.

### Chimeric Adenovirus 5 Fiber Vectors

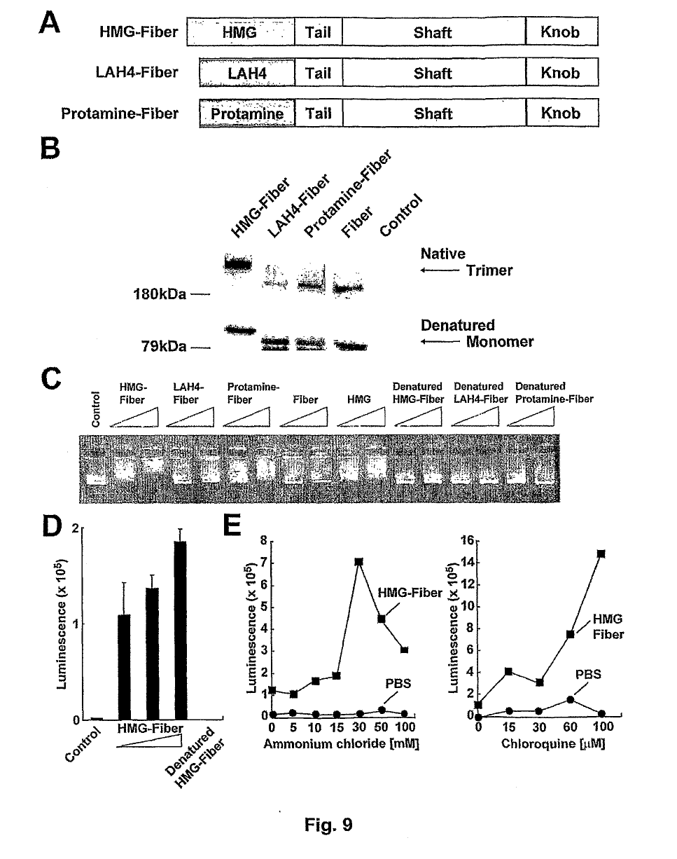

To determine whether DNA binding domains modified to include Fiber protein from adenovirus could improve binding to plasmid DNA, adenovirus type 5 (Ad5) Fiber, including the SV40 nuclear localization signal in the N terminal Fiber tail region, was fused to the DNA binding domain from high-mobility group box 1 (HMGB1; GenBank BC003378) box A domain, a cationic amphipathic histidine-rich peptide sequence, LAH4 (Kichler et al. 2006 Biochimica et Biophysica Acta 1758:301-307), or protamine (GenBank NP_{—}002752) (FIG. 9A). When these chimeric Fiber plasmids were transfected into 293T cells followed by immunoblotting analysis they were expressed and formed trimers (FIG. 9B). These modified DNA binding domains were then tested to measure their ability to bind to supercoiled plasmid. The purified chimeric proteins were mixed with plasmid and the products analyzed on 1% agarose gels. The plasmid bands associated with the HMG-Fiber construct showed the highest level of binding, as demonstrated by the shift in the bands. LAH4-Fiber and Protamine-Fiber constructs also bound, although with lower efficiency. Fiber alone or denatured chimeric Fibers did not result in any shift of the bands (FIG. 9C). Since the HMG-Fiber bound the plasmid most efficiently, we performed a study to determine whether the HMG-Fiber protein/plasmid complexes were able to deliver the plasmid to 293T cells. The HMG-Fiber was pre-incubated with luciferase reporter plasmid and the mixture was added dropwise to the 293T cells. As shown in FIG. 9D, luciferase activity was detected after transfection with HMG-Fiber/plasmid complex, but not after transfection with plasmid alone (control) or denatured HMG-Fiber/plasmid complex. We then investigated whether this complex was able to escape the endosomal compartment, because adenovirus is delivered to the endosome after entry into cells. The endosome was disrupted by treatment with the endosomal inhibitors, NH₄Cl or chloroquine, and the plasmid with HMG-Fiber was transfected into 293 cells. This treatment increased luciferase activity ˜10-fold, suggesting that endosome disruption increased the transfection efficiency.

Referring to FIG. 9, plasmid was delivered to cells by chimeric adenovirus 5 Fiber vector. A schematic representation of the chimeric adenovirus 5 Fiber vector is shown in FIG. 9A. The adenoviral protein Fiber was fused to DNA binding motif from HMGB-1 box A, LAH4 or protamine in the N terminus (HMG-Fiber, LAH4-Fiber or Protamine-Fiber, respectively). The His tag and SV40 nuclear localization signal are in front of each DNA binding motif. Chimeric Fiber proteins formed trimers (FIG. 9B). The chimeric adenovirus 5 Fiber, HMG-Fiber, LAH4-Fiber, Protamine-Fiber and WT Fiber were expressed from 293T cells and formed trimer. After 48 hours, the cell lysates with or without boiling were immunoblotted with monoclonal Fiber antibody. Chimeric Fiber bound to supercoiled DNA (FIG. 9C). No protein (Lane 1), HMG-Fiber 0.5 μg (Lane 2), HMG-Fiber 1.0 μg (Lane 3), LAH4-Fiber 0.5 μg (Lane 4), LAH4-Fiber 1.0 μg (Lane 5), Protamine-Fiber 0.5 μg (Lane 6), Protamine-Fiber 1.0 μg (Lane 7), Fiber 0.5 μg (Lane 8), Fiber 1.0 μg (Lane 9), HMG 0.5 μg (Lane 10), HMG 1.0 μg (Lane 11), Denatured HMG-Fiber 0.5 μg (Lane 12), Denatured HMG-Fiber 1.0 μg (Lane 13), Denatured LAH4-Fiber 0.5 μg (Lane 14), Denatured LAH4-Fiber 1.0 μg (Lane 15), Denatured Protamine-Fiber 0.5 μg (Lane 16), Denatured Protamine-Fiber 1.0 μg (Lane 17) were mixed with 0.5 μg of supercoiled plasmid for 30 min on ice, followed by resolution on 1% agarose gels. Agarose gels were stained with ethidium bromide. Chimeric Fiber was delivered via plasmid to 293T cells (FIG. 9D). 293T cells were transfected by the HMG-Fiber protein/DNA complex. HMG-Fiber 0 μg (Lane 1), HMG-Fiber 1.0 μg (Lane 2), HMG-Fiber 1.5 μg (Lane 3), HMG-Fiber 2.5 μg (Lane 4), Denatured HMG-Fiber 2.5 μg (Lane 5) were pre-incubated for 20 min at 37° C. with 0.5 μg of luciferase reporter plasmid before transfection. The mixture was directly transfected to 293T cells (5×10⁴) containing 2 ml DMEM medium in 48 well plates. Luciferase gene expression was measured 36 hours after transfection. HMG-Fiber protein/plasmid complex was transfected efficiently with treatment of lysosomal inhibitors (FIG. 9E). HMG-Fiber protein/DNA complex were transfected efficiently with NH₄Cl (left) and chloroquine (right). 2.5 μg of HMG-Fiber was pre-incubated for 30 min at 37° C. with 0.5 μg of luciferase reporter plasmid. The mixture was directly transfected to 293T cells (5×10⁴) containing 2 ml DMEM medium in 48 well plates and incubated for 30 min. The medium and the protein/DNA complex was removed and replaced with fresh medium. Then the cells were treated with the indicated amounts of lysosomal inhibitors for 30 min. The medium was removed and replaced with fresh medium. Luciferase gene expression was measured 36 hours after transfection.

### Chimeric Adenovirus 5 Penton Base Plus Fiber Vectors

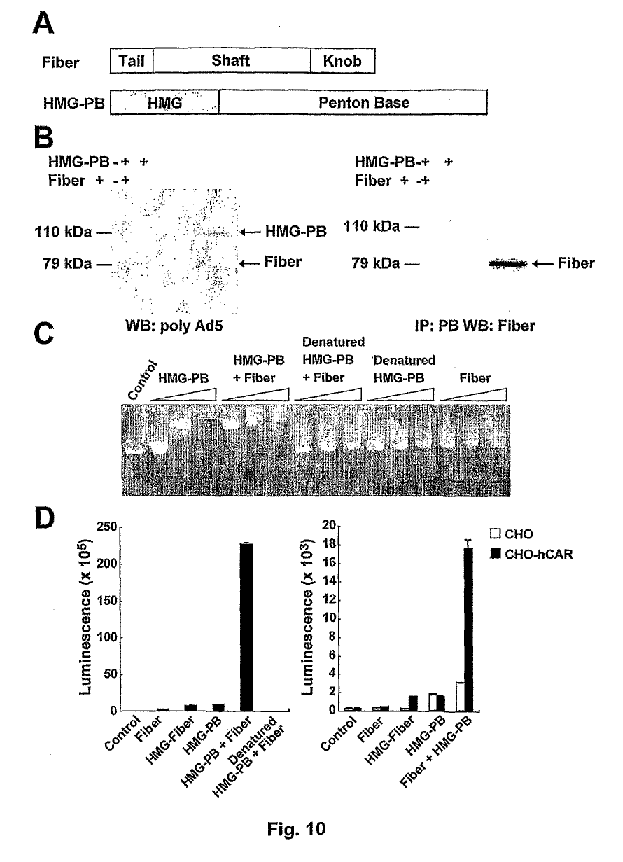

Since the penton base was implicated in disrupting the endosome, we developed another construct, a chimeric penton base (HMG-PB) vector designed to overcome the endosomal degradation of the protein/plasmid complex (FIG. 10A). The integrity of the HMG-PB/Fiber complex was confirmed by co-transfection of 293T cells with HMG-PB/Fiber followed by immunoprecipitation with HMG-PB and immunoblotting with Fiber (FIG. 10B, right). The gel shift assay was performed to determine whether the capacity of the HMG-PB/Fiber complex to bind to plasmid was retained. HMG-PB bound to the plasmid (FIG. 10C, lanes 2-4). Equal amounts of HMG-PB in either HMG-PB (FIG. 10C, Lane 3) or HMG-PB/Fiber complex (FIG. 10C Lanes 5-7) were run on an agarose gel. The supershift of the HMG-PB/Fiber complex indicated that these heteromeric proteins bind to the plasmid. The plasmid-bound complexes were then transfected into 293T cells to compare transfection efficiency. HMG-Fiber, and the HMG-PB/Fiber complex both delivered the plasmid approximately 16 times more efficiently (FIG. 10D, left). The luciferase plasmid with HMG-PB/Fiber complex was transfected into Chinese hamster ovary (CHO) cells expressing CAR (CHO-hCAR) and parental CHO cells (FIG. 10D, right). HMG-Fiber delivered plasmid only to CHO-hCAR cells but not the parental CHO cells, suggesting that entry was dependent on the interaction of CAR with the Fiber. In contrast, HMG-PB delivered the plasmid to both the CHO-hCAR and CHO cells, presumably through the interaction of αV integrin and the RGD motif in the penton base. The HMG-PB/Fiber complex delivered plasmid approximately 10 times more efficiently to CHO-hCAR cells compared to HMG-Fiber or HMG-PB alone, suggesting a synergistic response. The HMG-PB/Fiber complex delivered plasmid about 6 times more efficiently to CHO-hCAR cells compared to CHO cells, suggesting that entry is dependent on Fiber binding to CAR.

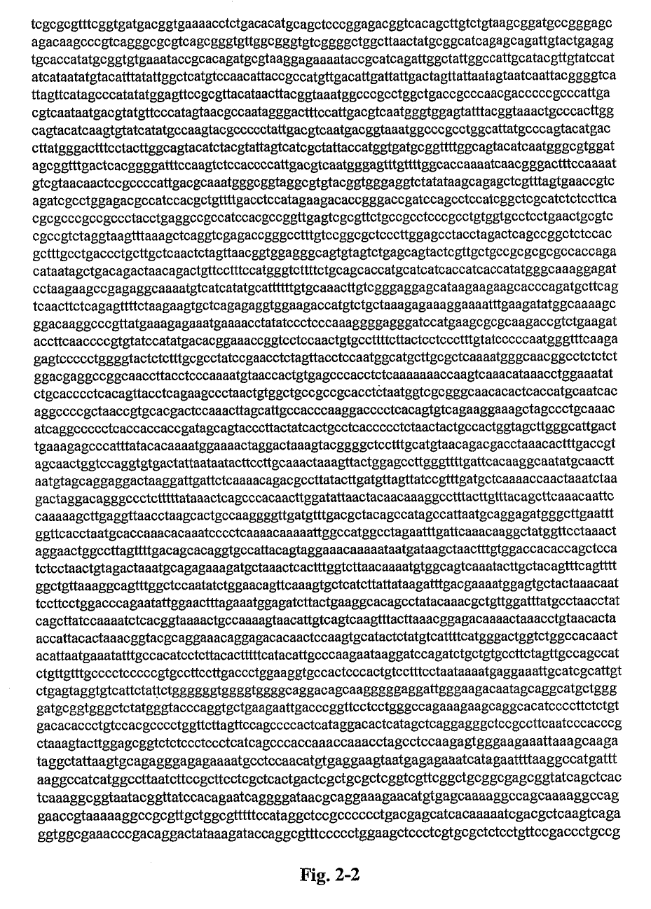

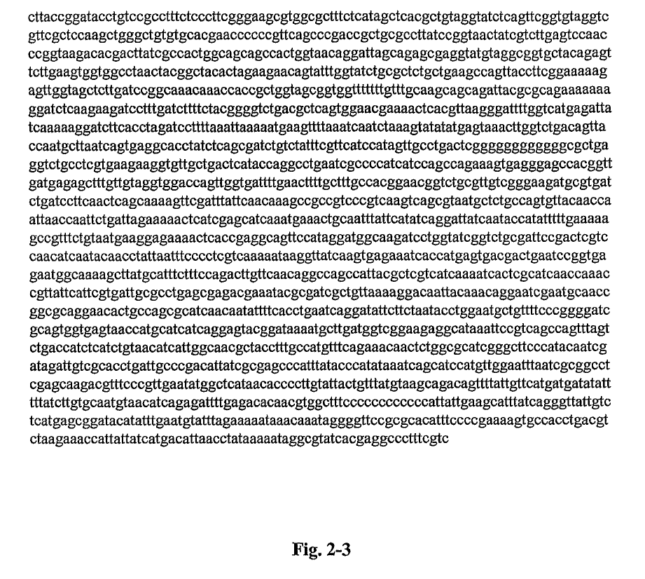

Referring to FIG. 10, plasmid was delivered to cells by chimeric adenovirus 5 Fiber and Penton Base vectors. A schematic representation of chimeric adenovirus 5 Penton Base plus Fiber vector is shown in FIG. 10A. The Fiber was fused to a His tag in the C terminus with a GS linker. The adenoviral protein Penton Base was fused to a DNA binding motif from the HMGB-1 box A in the N terminus. The His tag and SV40 nuclear localization signal is in front of HMGB-1 box A. HMG-PB bound to Fiber is shown in FIG. 10B. The HMG-PB and Fiber (Fiber without His tag) were expressed from 293T cells and the HMG-PB bound to Fiber. The indicated plasmids (1 μg each) were transfected into 293T cells. After 48 hours, the cell lysates were immunoblotted with polyclonal Ad5 antibodies (left). The cell lysates were immunoprecipitated with His antibody (Penton Base) and immunoblotted with Fiber monoclonal antibody (right). Chimeric Penton Base and Fiber bound to supercoiled DNA (FIG. 10C). No protein (Lane 1), HMG-PB 0.5 μg, 1.0 μg and 1.5 μg (Lanes 2-4), HMG-PB 1.0 μg plus Fiber 0.5 μg, HMG-PB 1.0 μg plus Fiber 1.0 μg and HMG-PB 1.0 μg plus Fiber 1.5 μg (Lanes 5-7), denatured HMG-PB 1.0 μg plus Fiber 0.5 μg, HMG-PB 1.0 μg plus Fiber 1.0 ρg and HMG-PB 1.0 μg plus Fiber 1.5 μg (Lanes 8-10), denatured HMG-PB 0.5 μg, 1.0 μg and 1.5 μg (Lanes 11-13) and Fiber, 0.5 μg, 1.0 μg and 1.5 μg (Lanes 14-16) were mixed with 0.5 μg of supercoiled plasmid for 30 min on ice, followed by resolution on 1% agarose gels. Agarose gels were stained with ethidium bromide. Chimeric penton base and Fiber were delivered via plasmid to 293T and CHO-hCAR cell line (FIG. 2D). 293T cells, CHO cells and human coxsackie virus B adenovirus receptor expressing CHO cells (CHO-hCAR) were transfected by the HMG-PB plus Fiber protein/DNA complex. The indicated proteins were pre-incubated for 20 min at 37° C. with 0.5 μg of luciferase reporter plasmid before transfection. The mixture was directly transfected to 293T cells (5×10⁴) containing 2 ml DMEM medium in 48 well plates. Luciferase gene expression was measured 36 hours after transfection.

Because adenovirus is thought to be transported by dynein/dynactin cytoskeletal components to nuclear microtubules after endosomal escape, Kelkar et al. 2004 J Virology 78:10122-10132; Meier, O. and Greber, U. F 2003 J Gene Med 5:451-462), HMG-PB was modified to add the human herpesvirus dynein binding motif (Martinez-Moreno et al. 2003 FEBS letters 544:262-267) between the C-terminal HMG binding motif and the N-terminal Penton Base (HMG-dynein-PB) to increase efficiency of the transfer of plasmid with chimeric protein complex to the nucleus. The reporter luciferase plasmid containing HMG-dynein-PB/Fiber transfected 293T cells at a level that was 2-4 times lower than the plasmid with HMG-PB/Fiber complex. This finding suggested that alternative motifs or positions of analogous motifs might be required for dynein targeting.

To increase the efficiency of plasmid DNA, the penton base was fused to the complete DNA binding domain of HMG-1, containing -not only the box A domain used previously but also including the box B domain (HMGfull-PB). The plasmid with HMGfull-PB/Fiber complex was delivered to 293T cells approximately 2-3 times more efficiently than the plasmid with HMG-PB/Fiber complex.

### Chimeric Adenovirus 5 Protein V Synthesis Vectors

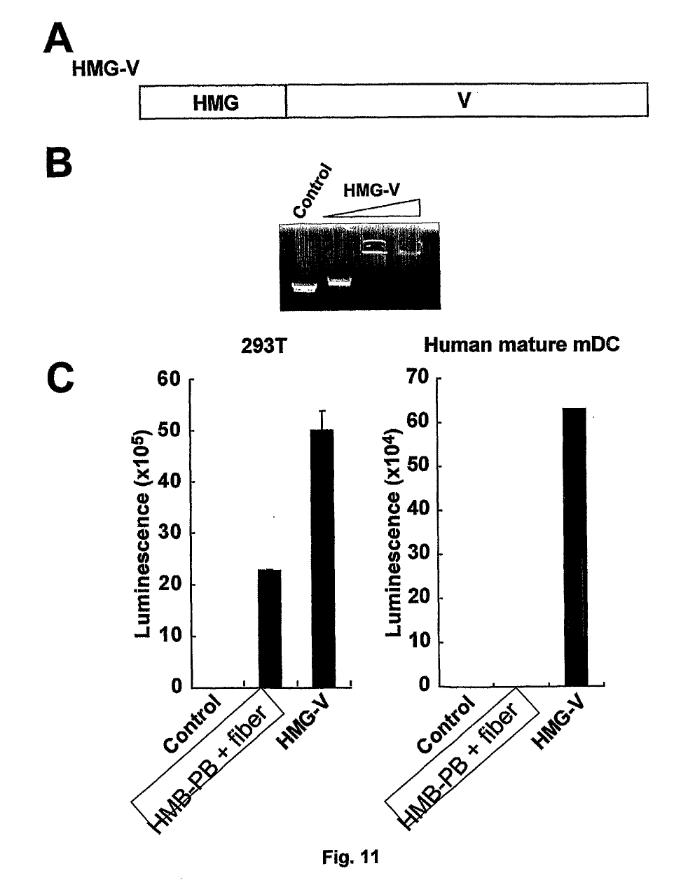

The DNA core of adenovirus is covered by a core complex largely composed of protein V, thought potentially to facilitate transport to the nucleus through the nuclear pore complex. To stimulate this process, we developed an alternative adenoviral chimera, protein V vector fused to the HMG box A domain (HMG-V), to protect the plasmid in the cytosol and to increase the transfer of the plasmid to the nucleus (FIG. 11A). HMG-V bound to the plasmid efficiently as demonstrated in the gel shift assay (FIG. 11B). The luciferase plasmid with HMG-V was transfected into 293T and dendritic cells (DC). Compared to the plasmid complexed to HMG-PB/Fiber, the plasmid with HMG-V was delivered approximately 2 times more efficiently (FIG. 11C, left). Since the protein V is highly basic, the plasmid/HMG-V complex could potentially enter cells through a specific receptor interaction. The plasmid with HMG-V was delivered efficiently to human DC cells, while the plasmid with HMG-PB/Fiber complex was not delivered to these cells (FIG. 11C, right).

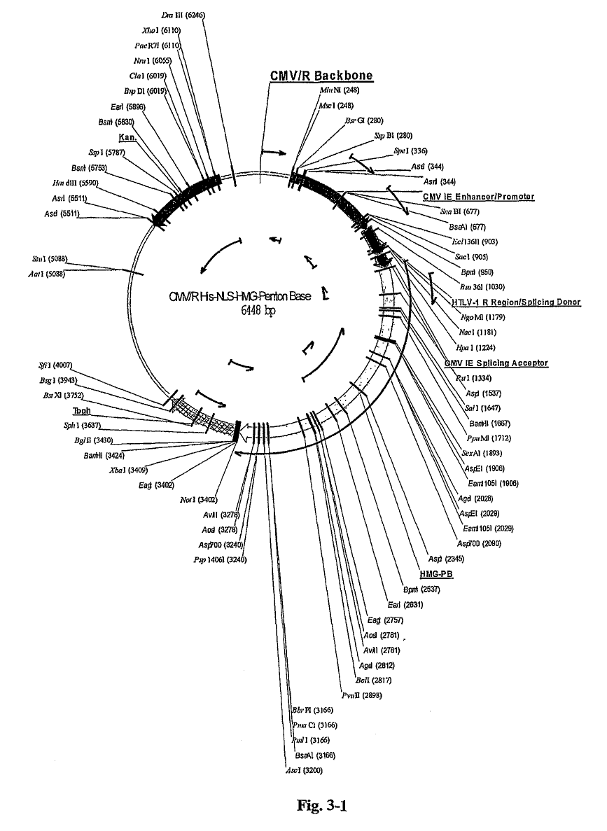

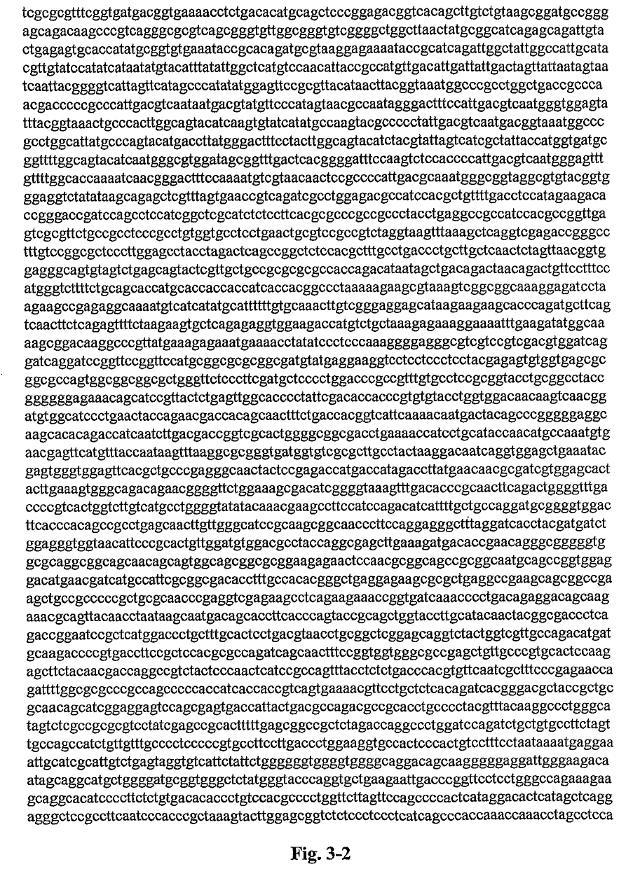

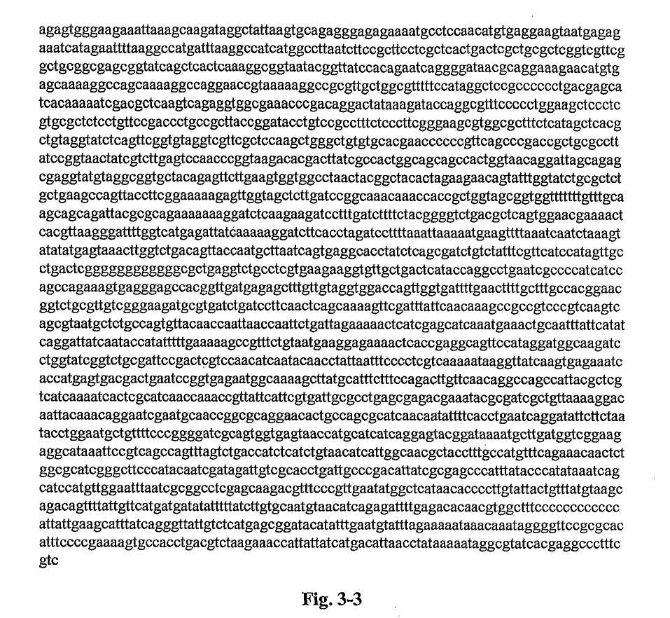

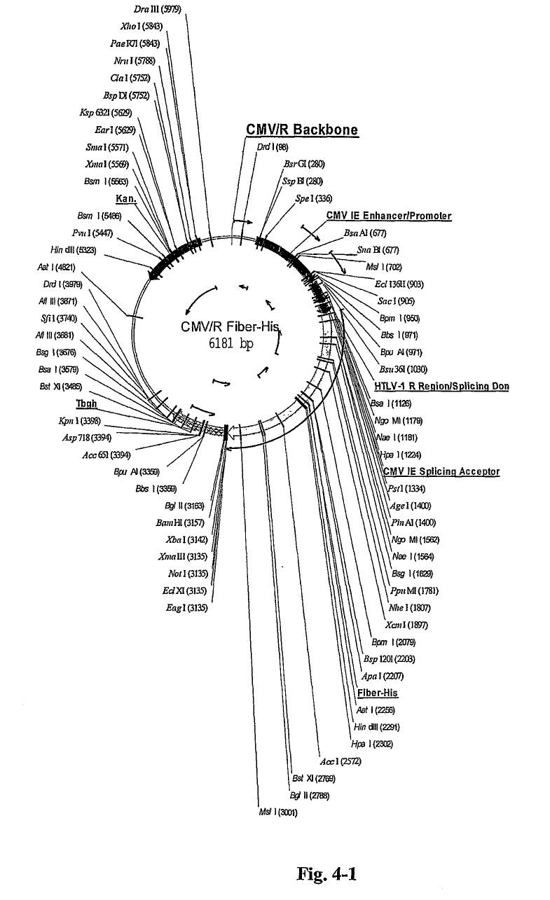

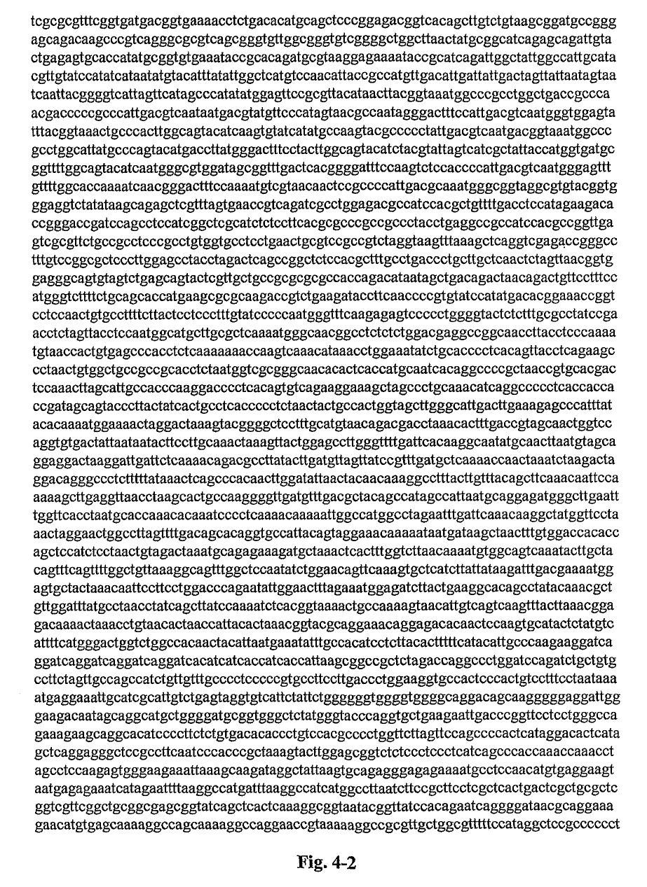

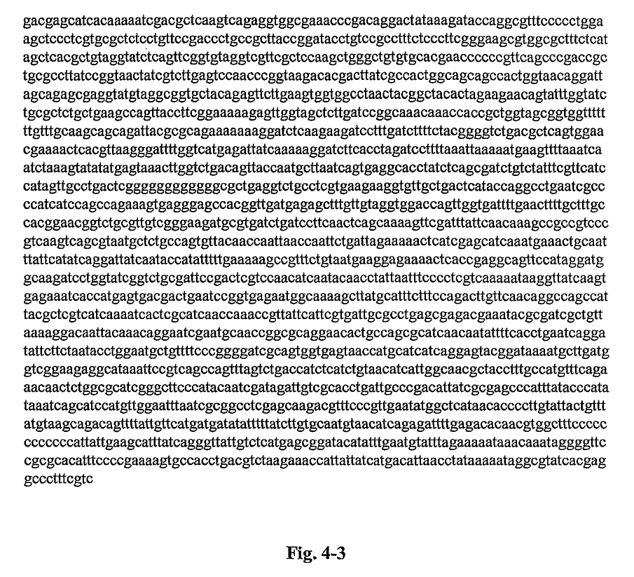

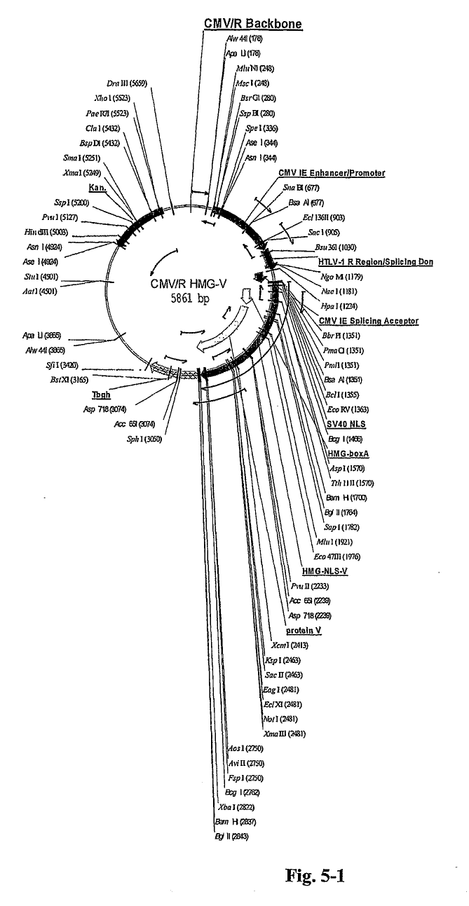

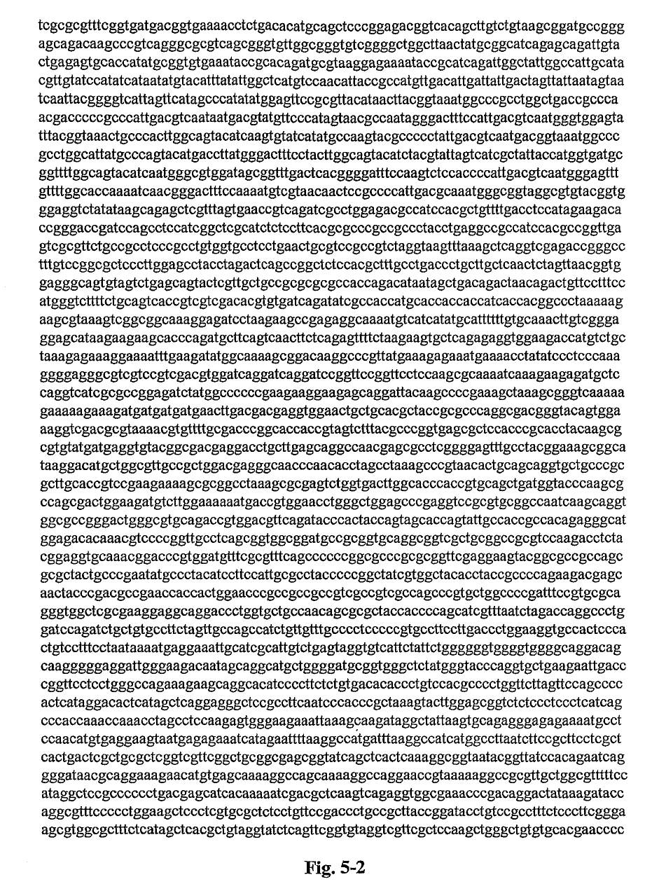

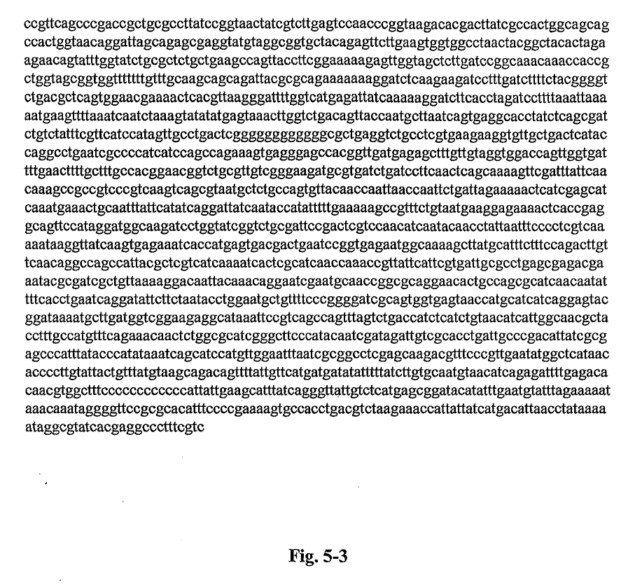

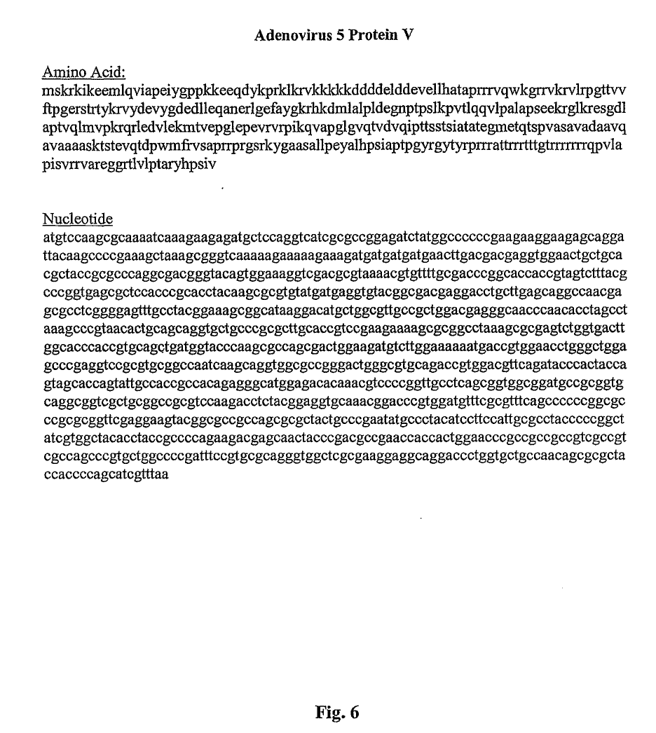

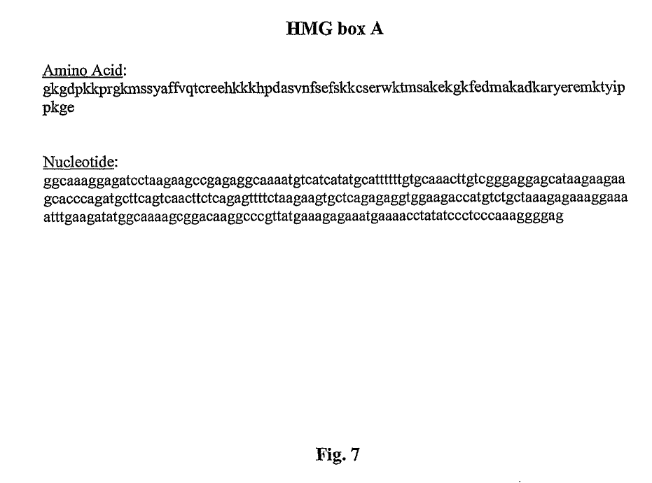

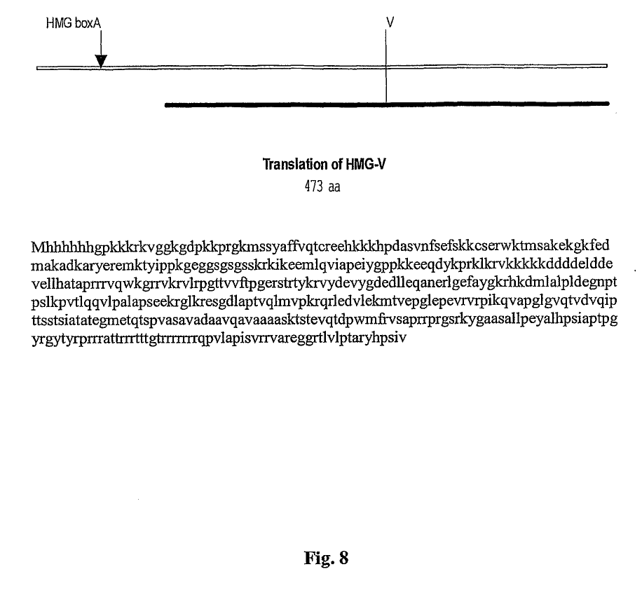

Referring to FIG. 11, plasmid was delivered to cells by chimeric adenovirus 5 V vector. A schematic representation of the chimeric adenovirus 5 protein V vector is shown in FIG. 3A. The adenoviral protein V was fused to the DNA binding motif from HMGB-1 box A in the N terminus. The His tag and SV40 nuclear localization signal is in front of HMGB-1 box A. Chimeric HMG-V bound to supercoiled DNA (FIG. 3B). No protein (Lane 1), HMG-V 0.5 μg (Lane 2), HMG-V 1.0 μg (Lane 3) and HMG-V 1.5 μg (Lane 4) were mixed with 0.5 μg of supercoiled plasmid for 30 min on ice, followed by resolution on 1% agarose gels. Agarose gels were stained with ethidium bromide. Chimeric HMG-V delivered via plasmid to 293T cells and human mature myeloid DC (mDC) (FIG. 11C). 293T (left) and mDC (right) cells were transfected with the indicated protein/DNA complex. mDCs were isolated by counterflow centrifugal elutriation followed by magnetic bead isolation (Miltenyi Biotec) using CD1c isolation kits. mDCs were cultured in a complete medium of RPMI 1640 supplemented with 1% streptomycin and penicillin, 10% FCS (Invitrogen) and recombinant human GM-CSF (2 ng/ml; PeproTech). Poly (I:C) was added for the maturation 24 hours after isolation and mDC were transfected after 6 days culture. The indicated proteins were pre-incubated for 20 min at 37° C. with 0.5 μg (293T) or 2.0 μg (mDC) of luciferase reporter plasmid before transfection. The mixture was directly transfected to 293T cells (5×10⁴) in 48 well plates or DC (5×10⁴) in 96 well plates. Luciferase gene expression was measured 24 hours after transfection.

### Injection of Mice with HIV-1 Plasmid with HMG-V

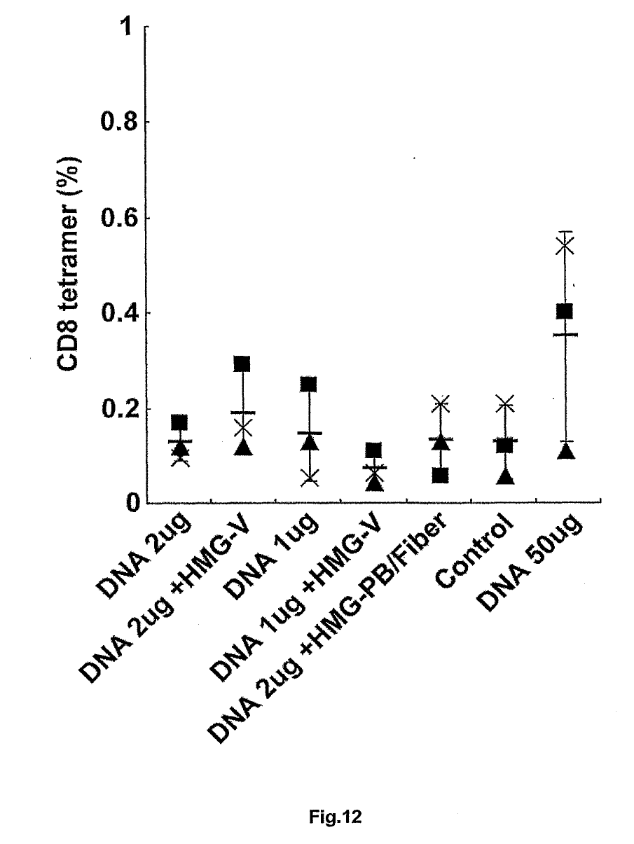

The HIV-1 envelope (Env) plasmid with HMG-V was injected into mice, and 14 days after injection, the CD8 T cell response against HIV-1 from blood PBMC was measured using a V3 p18-I10 peptide tetramer assay (FIG. 12). Because of the limitation of the concentration of HMG-V, the mice were injected with small quantities. The mice injected with plasmid and HMG-V complex did not show an increase in the CD8 T cell response compared to the mice injected with the plasmid alone. We envision formulating the HMG-V to allow for titration of the response with larger quantities.

Referring to FIG. 12, chimeric HMG-V/plasmid HIV-1 Env plasmid complex was injected into mice. The plasmid HIV-1 Env Hxbc2 expression vector was mixed with the chimeric HMG-V protein at 37° C. for 20 min. The mice were injected with the indicated amount of plasmid alone or chimeric HMG-V/plasmid complex. Fourteen days after injection, PBMCs from tail vein blood were analyzed using a V3 peptide RGPGRAFVTI (SEQ ID NO: 42) tetramer with CD8a (Ly-2) antibody (BD Pharmingen).

### Injection of Mice with Effective Quantities of HIV-1 Plasmid with HMG-V

The HIV-1 envelope (Env) plasmid with HMG-V is injected in effective quantities into mice, and 14 days after injection, the CD8 T cell response against HIV-1 from blood PBMC is measured using a V3 p18-I10 peptide tetramer assay. The mice injected with the effective quantities of plasmid and HMG-V complex demonstrate a statistically significant increase in the CD8 T cell response compared to the mice injected with the plasmid alone.

### Discussion

In this disclosure, we have shown that plasmid complexed with adenoviral protein components improved delivery of DNA into different cells, including antigen presenting cells (APC). To date, there has been no highly effective method to efficiently transfer plasmid DNA into APC cells. Use of the chimeric HMG-V protein is anticipated to provides an improvement over the more standard, though much less efficient, liposome-based transfection method.

The HMG-Fiber and HMG-PB/Fiber complexes were able to deliver plasmid to 293T cells but not to DC cells. The HMG-Fiber and the HMG-PB/Fiber complex bound to the cellular receptor, although, once attached, it is not necessarily transported to the nucleus and degraded in the cytosol. By comparison, HMG-V efficiently delivered plasmid to DC cells, although the mechanism of the delivery is not completely understood. Because protein V has a highly basic charge, the plasmid/HMG-V complex entered the cell non-specifically. There is some evidence to suggest that protein V might play an important role in protecting the plasmid as a particle form and transporting the plasmid to the nucleus efficiently. We envision that the findings presented in this disclosure provide the basis for improved delivery efficiency for DNA vaccines.

### EXAMPLE 1

### Construction of the Chimeric Adenoviral Vector

Each adenoviral Fiber, Penton Base and V gene was amplified by the polymerase chain reaction (PCR) (Einfeld, D. A. et al. 2001 J Virol 75:11284-11291, primers set forth in Table 4) from human adenovirus type 5 (Genbank AC000008). The DNA binding domain from the HMG box A, LAH4 and Protamine gene with N terminal His-tag and SV40 nuclear localization signal was amplified with synthesized oligonucleotides (see Table 2) by PCR. The Fiber fragment was digested with AgeI and NotI and DNA binding domain fragments were digested with PstI and AgeI and cloned into the CMV/R vector (Yang, Z.-Y. et al. 2004 J Virol 78:5642-5650) digested with PstI and NotI (HMG-Fiber, LAH4-Fiber and Protamine-Fiber, respectively). The fragment of the HMG box A amplified by PCR was digested with PstI and BamHI, and the fragment of Penton Base amplified by PCR was digested with BamHI and XbaI. These two fragments were inserted into the CMV/R vector digested with PstI and XbaI (HMG-PB). The fragment of dynein binding motif amplified by PCR was digested with BspEI and BamHI. The fragment of HMG box A amplified by PCR was digested with PstI and BspEI. These two fragments were subcloned into the CMV/R vector. The Penton Base fragment digested with BamHI and XbaI and the HMG-dynein fragment were inserted into the CMV/R vector (HMG-dynein-PB). The fragment of HMG box A and box B amplified by PCR was digested with PmeI and BgIII, and the fragment of Penton Base amplified by PCR was digested with BamHI and XbaI. These two fragments were inserted into the CMV/R vector digested with EcoRV and XbaI (HMGfull-PB).

TABLE 4
Polymerase Chain Reaction Primers

The HMG box A domain was amplified with:
5′-CTGCAGCACCATGCATCATCACCATCACCATATGGGCAAAGGAGA
TCCTA-3′, (SEQ ID NO: 10);
5′-CATATGATGACATTTTGCCTCTCGGCTTCTTAGGATCTCCTTTGC
CCATA-3′, (SEQ ID NO: 11);
5′-AGGCAAAATGTCATCATATGCATTTTTTGTGCAAACTTGTCGGGA
GGAGC-3′, (SEQ ID NO: 12);
5′-TGACTGAAGCATCTGGGTGCTTCTTCTTATGCTCCTCCCGACAAG
TTTGC-3′, (SEQ ID NO: 13);
5′-GCACCCAGATGCTTCAGTCAACTTCTCAGAGTTTTCTAAGAAGTG
CTCAG-3′, (SEQ ID NO: 14);
5′-TCTCTTTAGCAGACATGGTCTTCCACCTCTCTGAGCACTTCTTAG
AAAAC-3′, (SEQ ID NO: 15);
5′-GACCATGTCTGCTAAAGAGAAAGGAAAATTTGAAGATATGGCAAA
AGCGG-3′, (SEQ ID NO: 16);
5′-TTTTCATTTCTCTTTCATAACGGGCCTTGTCCGCTTTTGCCATAT
CTTCA-3′, (SEQ ID NO: 17);
5′-TTATGAAAGAGAAATGAAAACCTATATCCCTCCCAAAGGGGAGGG
ATCCA-3′, (SEQ ID NO: 18);
5′-AGGTATCTTCAGACGGTCTTGCGCGCTTCATGGATCCCTCCCCTT
TGGGA-3′, (SEQ ID NO: 19);
5′-AAGACCGTCTGAAGATACCTTCAACCCCGTGTATCCATATGACAC
GGAAA-3′, (SEQ ID NO: 20); and
5′-GAGTAAGAAAAGGCACAGTTGGAGGACCGGTTTCCGTGTCATATG
GATAC-3′(SEQ ID NO: 21).
The LAH4 domain was amplified with:
5′-AAAAGTCGACCACTAAACGGTACACAGGAAACAGGGTCTAGAGGA
TTTAAATCTGGATCCTACCCCTACGACGTG-3′,
(SEQ ID NO: 22);
5′-GAAAATGACATAGAGTATGCACTTGGAGTTGTGTCGCCGGCGTAG
TCGGGCACGTCGTAGGGGTAGGATCCAGAT-3′,
(SEQ ID NO: 23);
5′-CAAGTGCATACTCTATGTCATTTTCATGGGACTGGTCTGGCCACA
ACTACATTAATGAAATATTTGCCACATCCT-3′,
(SEQ ID NO: 24;
5′-GAGCAGAGCTTTCTTACTGCTTTCTTGGGCAATGTATGAAAAAGT
GTAAGAGGATGTGGCAAATATTTCATTAAT-3′,
(SEQ ID NO: 25);
5′-AGAAAGCAGTAAGAAAGCTCTGCTCGCCCTGGCTTTGCACCATCT
TGCTCATCTCGCCTTGCATCTTGCTCTTGC-3′, (SEQ ID NO: 26)
and
5′-TTGCGGCCGCTCAATGGTGATGGTGATGATGACTACCAGCCTTCT
TCAGTGCAAGAGCAAGATGCAAGGCGAGAT-3′(SEQ ID NO: 27).
The Protamine domain was amplified with:
5′-CTGCAGCACCATGCATCATCACCATCACCATATGGCCAGGTACAG
ATGCTGTCGCAGCCAGAGCCGGAGCAGATATTACCGCCAG-3′,
(SEQ ID NO: 28);
5′-CTCCTCCGTGTCTGGCAGCTCCGCCTCCTTCGTCTGCGACTTCTT
TGTCTCTGGCGGTAATATCTGCTCCGGCTC-3′, (SEQ ID NO: 29);
5′-GGCGGAGCTGCCAGACACGGAGGAGAGCCATGAGGTGCTGCCGCC
CCAGGTACAGACCGAGATGTAGAAGACACG-3′, (SEQ ID NO: 30)
and
5′-GACCGGTTTCCGTGTCATATGGATACACGGGGTTGAAGGTATCTT
CAGACGGTCTTGCGCGCTTCATGGATCCGTGTCTTCTACATCTCGGTC
TGTA-3′(SEQ ID NO: 31).
The Penton Base domain was amplified by:
5′-AAAGGATCCGGTTCCGGTTCCATGCGGCGCGCGGCGATGTATG-3′
(SEQ ID NO: 32) and
5′-TAAATCTAGATTAAAAAGTGCGGCTCGATAGGACGCGCG-3′
(SEQ ID NO: 33).
The Protein V domain was amplified by:
5′-GGATCCGGTTCCGGTTCCTCCAAGCGCAAAATCAAAGA-3′
(SEQ ID NO: 34) and
5′-TTTCTAGATTAAACGATGCTGGGGTGGTAGCGCGC-3′
(SEQ ID NO: 35).
The dynein domain was amplified by:
5′-GGGCTCCGGAGTGACCATTCTGGTGAGCCG-3′,
(SEQ ID NO: 36);
5′-CCAGGCCGGTCTGGGTGCTGCGGCTCACCA-3′,
(SEQ ID NO: 37);
5′-ACCGGCCTGGGCCATTTTACCCGCAGCACC-3′,
(SEQ ID NO: 38);
5′-ATATCGTTCTGGCTGGTCTGGGTGCTGCGG-3′,
(SEQ ID NO: 39);
5′-AGAACGATATTTTTGTCGTCCGTCGACGTG-3′
(SEQ ID NO: 40) and
5′-ACCGGATCCTGATCCTGATCCACGTCGACG-3′
(SEQ ID NO: 41).

The fragment of HMG box A amplified by PCR was digested with EcoRV and BamHI, and the fragment of V amplified by PCR was digested with BamHI and XbaI. These two fragments were inserted into CMV/R vector digested with EcoRV and XbaI (HMG-V).

### Purified Chimeric Adenoviral Protein

The 293T cells were transfected with 16 μg of chimeric adenoviral plasmid in each 15 cm plate by using a calcium phosphate transfection kit (Promega) or a FuGENE™ 6 Transfection Reagent kit (Roche Diagnostics GmbH, Germany) according to the manufacturer's recommendations. Forty eight hours after transfection, cells were lysed by cell lysis buffer (Cell signaling technology) and incubated with Ni-NTA resins agarose (Qiagen) in 20 mM sodium phosphate, 500 mM NaCl and 30 μM imidazole overnight at 4° C. Proteins were eluted with 20 mM sodium phosphate, 500 mM NaCl and 200 μM imidazole buffer. Proteins were purified with a PD10 column (Amersham Biosciences) in PBS buffer.

### Immunoblotting and Immunoprecipitation

The immunoblotting and immunoprecipitation assays were performed as previously described (Akahata W. et al. 2005 J Virol 79:626-631; and Ganesh, L. et al. 2003 Nature 426:853-857). Antibodies against mouse-adeno Fiber antibody 4D2 (Biocompare, AB3233), anti-His antibody (Invitrogen, R940-25), anti-adenovirus type 5 polyclonal antibody (Abcom, AB6982) and goat anti-rabbit or mouse IgG-HRP (Santa Cruz Biotechnology, sc-2054 and sc-2005, respectively) as a 2nd antibody were used following the manufacture's instructions.

### Transfection and Luciferase Assay

Chimeric adenoviral proteins were incubated in PBS with 0.5 μg of CMV/R luciferase plasmid to 293T cells or 2 μg of the luciferase plasmid to human DC for 20 min at 37° C. The mixture was added dropwise to 1×10⁵ 293T cells in 1 ml DMEM medium in a 48 well plate, or 1×10⁵ mature DC in 100 μl RPMI medium containing GM-CSF in a 98 well plate. Forty eight hours after transfection, luciferase activity in cell lysates was measured per the manufacturer's instructions (Promega) using a Top Count luminometer (Packard).

### DC Isolation and Cell Culture

Human myeloid DC (mDC) were isolated as described previously (Ganesh, L. et al. 2004 J Virol 78:11980-11987). Briefly, mDCs were isolated from the elutriated monocyte fraction by deletion of cells expressing BDCA-4 and CD19 by using microbeads (Miltenyi Biotech, Auburn, Calif.) and positive selection by using antibodies to CDlc (Miltenyi Biotech) followed by RPMI culture supplemented with 10% heat-inactivated fetal bovine serum (FBS) (Gibco BRL) and granulocyte-macrophage colony-stimulating factor (10 ng/ml; PeproTech, Rocky Hill, N.J.). To initiate differentiation of DCs, cells were treated with poly (I:C) (50 ng/ml; Sigma) for 2-6 days.

### Mouse Immunization and Tetramer Assay

Female 6- to 8-week-old BALB/c mice were injected in the right and left quadriceps muscles with 1, 2 or 10 μg of purified plasmid HIV-1 HXBc2 Env (Cayabyab et al. 2006 J Virol 80:1645-1652) with chimeric adenoviral protein (10 μg) suspended in 100 μP of normal saline in the quadriceps muscles. Each group of five mice was injected. Fourteen days after injection, blood was collected from the tail vein and a tetramer assay was performed as previously described (Seaman et. al. 2004 J Virology 78:206-215). Statistical analysis was performed by two-tail distribution paired t-test. Animal experiments were conducted in compliance with all relevant federal guidelines and NIH policies.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. A composition comprising a gene delivery fusion protein (GDFP), joined to a gene delivery domain (GDD), wherein said GDFP comprises a nucleic acid binding domain (NBD) and said GDD comprises an adenovirus protein V or derivative thereof that retains protein V activity.

2. The composition according to claim 1, wherein the NBD interacts with a double-stranded nucleic acid.

3. The composition according to claim 1, wherein the NBD interacts with a single-stranded nucleic acid.

4. The composition according to claim 1, wherein the NBD interacts with a DNA or an analog thereof.

5. The composition according to claim 1, wherein the NBD interacts with a RNA or an analog thereof.

6. The composition according to claim 1, wherein the NBD interacts with a recombinant expression vector.

7. The composition according to claim 1, wherein said GDFP further comprises a flexible polypeptide linker sequence.

8. The composition according to claim 1, wherein said NBD comprises a nucleic acid binding component of a sequence-specific nucleic acid binding protein.

9. The composition according to claim 1, wherein said NBD comprises a nucleic acid binding component of a sequence-non-specific nucleic acid binding protein.

10. The composition according to claim 1, wherein said GDD comprises two or more components selected from the group consisting of a binding component, a membrane-disrupting component, and a localization component.

11. The composition of claim 1, further comprising a nucleic acid joined to said NBD.

12. A recombinant polynucleotide encoding the composition of claim 1.

13. The recombinant polynucleotide of claim 12, further comprising an expression vector that comprises a transcriptional control region operably linked to said recombinant polynucleotide.

14. A cell that comprises the recombinant polynucleotide of claim 12.

15. A method of making the composition of claim 1 comprising:
providing a recombinant polynucleotide encoding the composition of claim 1 to cell under conditions that allow for the expression of said recombinant polynucleotide.

16. A method of using the composition of claim 1 to deliver a nucleic acid to a cell comprising:
providing the composition of claim 1 to a cell under conditions that allow for the binding of a nucleic acid to said NBD; and
measuring the amount of nucleic acid delivered to said cell.

17. A method of using the composition of claim 1 to stimulate an antigen-specific immune response, comprising:
providing the composition of claim 1 to an animal under conditions that allow for the binding of a nucleic acid to said NBD; and
measuring an immune response of said animal.

18. The composition of claim 1, wherein said composition comprises the sequence of SEQ. ID. No. 1.

19. The composition of claim 1, wherein said composition comprises the sequence of SEQ. ID. No. 2 or 3.

20. The composition of claim 1, wherein said composition comprises the sequence of SEQ. ID. No. 4.
